(19) United States
(12) Patent Application Publication
(10) Pub. No.: US 2026/0053416 A1
(43) Pub. Date: Feb. 26, 2026

(54) ELECTROENCEPHALOGRAPHY DRY ELECTRODE AND ELECTROENCEPHALOGRAPHY MEASUREMENT APPARATUS HAVING SAME

(71) Applicant: KINGFAR INTERNATIONAL INC., Beijing (CN)

(72) Inventors: Qichao ZHAO, Beijing (CN); Ran YANG, Beijing (CN)

(21) Appl. No.: 19/373,782

(22) Filed: Oct. 30, 2025

Related U.S. Application Data
(63) Continuation of application No. PCT/CN2024/136866, filed on Dec. 4, 2024.

(30) Foreign Application Priority Data
Dec. 5, 2023 (CN) 202311657227.9
Dec. 5, 2023 (CN) 202311657239.1
Dec. 5, 2023 (CN) 202311657918.9
Dec. 26, 2023 (CN) 202311810168.4
Dec. 26, 2023 (CN) 202311814246.8

(51) Int. Cl. A61B 5/291; A61B 5/263; A61B 5/369

(52) U.S. Cl. CPC A61B 5/291; A61B 5/263; A61B 5/369

(57) ABSTRACT

Provided are an electroencephalography dry electrode and an electroencephalography measurement apparatus having the same. The electroencephalography dry electrode includes a substrate and a plating layer. The substrate includes a base and a plurality of columnar members. An end of each of the plurality of columnar members is fixed to the base. At least a surface of each of the plurality of columnar members is provided with the plating layer. The plating layer on the surface of each of the plurality of columnar members is electrically connected to the base.

### CROSS-REFERENCE TO RELATED APPLICATION

This application is a continuation of International Patent Application No. PCT/CN2024/136866, filed on Dec. 4, 2024, which is based on Chinese patent application No. 202311810168.4, filed on Dec. 26, 2023, and titled “ELECTROENCEPHALOGRAPHY DRY ELECTRODE AND ELECTROENCEPHALOGRAPHY MEASUREMENT APPARATUS HAVING THE SAME”, Chinese patent application No. 202311814246.8, filed on Dec. 26, 2023, and titled “ELECTROENCEPHALOGRAPHY MEASUREMENT APPARATUS AND CONTROL METHOD OF SIGNAL ACQUISITION ELECTRODE THEREOF”, Chinese patent application No. 202311657227.9, filed on Dec. 5, 2023, and titled “ELECTROENCEPHALOGRAPHY MEASUREMENT APPARATUS, ACQUISITION METHOD, AND ACQUISITION CIRCUIT”, Chinese patent application No. 202311657239.1, filed on Dec. 5, 2023, and titled “ELECTROENCEPHALOGRAPHY MEASUREMENT APPARATUS, ACQUISITION METHOD, AND ACQUISITION CIRCUIT”, and Chinese patent application No. 202311657918.9, filed on Dec. 5, 2023, and titled “ELECTROENCEPHALOGRAPHY MEASUREMENT APPARATUS, ACQUISITION METHOD, AND ACQUISITION CIRCUIT”, and claims priorities to the above Chinese patent applications. The above patent applications are incorporated herein by reference in their entireties.

### FIELD

The present disclosure relates to the field of electroencephalography measurement technologies, and more particularly, to an electroencephalography dry electrode and an electroencephalography measurement apparatus having the same.

### BACKGROUND

Existing electrodes for electroencephalography measurement commonly use a type where a conductive paste is placed between an examinee's scalp and an electrode. In addition to reducing a contact impedance between the scalp and the electrode, the conductive paste also serves to fix a measurement site position. However, it is relatively cumbersome to use because the conductive paste needs to be removed after measurement.

Dry electrodes, on the other hand, can ensure a low contact impedance without the use of conductive paste. In the related art, a flat-plate dry electrode, with its conductive surface in direct contact with the skin, can adhere closely to the skin, but cannot be used in hairy areas.

### SUMMARY

The present disclosure aims to solve one of the technical problems in the related art. To this end, an objective of the present disclosure is to provide an electroencephalography dry electrode that can pass through the hair and contact the scalp, and can be used for electroencephalography measurement in hairy areas.

According to an embodiment of a first aspect of the present disclosure, the electroencephalography dry electrode includes a substrate and a plating layer. The substrate includes a base and a plurality of columnar members. An end of each of the plurality of columnar members is fixed to the base. At least a surface of each of the plurality of columnar members is provided with the plating layer. The plating layer on the surface of each of the plurality of columnar members is electrically connected to the base.

According to the electroencephalography dry electrode of the embodiment of the present disclosure, the plurality of columnar members are provided, and the end of each of the plurality of columnar members is fixed to the base. When it is needed to acquire an electroencephalography signal from a hairy area, the plurality of columnar members pass through the hair, in such a manner that the other ends of the plurality of columnar members contact the scalp. The plurality of columnar members in the electroencephalography dry electrode of the present disclosure can pass through the hair, in such a manner that the other ends of the plurality of columnar members can maintain contact with the scalp, resolving a problem in the related art that a flat-plate dry electrode cannot be used in the hairy areas. Further, by providing the plurality of columnar members, a contact area between the electroencephalography dry electrode and the scalp can be increased to reduce a contact impedance.

In some embodiments, each of the plurality of columnar members includes a first member, a second member, and a first elastic member. The first member is sleeved at the second member. The second member is configured to move relative to the first member in an axial direction. The first elastic member is disposed in the first member. Two ends of the first elastic member are respectively connected to the first member and the second member. The first elastic member is configured to apply a restoring force to the second member to move outward from the first member.

In some embodiments, each of the plurality of columnar members includes a copper member.

In some embodiments, the second member has a recessed cavity. The recessed cavity has a first opening facing towards the first elastic member. The first elastic member passes through the first opening with one end of the first elastic member located in the recessed cavity. The second member is formed by stamping and stretching.

In some embodiments, a limit portion is disposed at a circumferential edge of the first opening, and has a dimension greater than an outer diameter of the second member in a direction perpendicular to an axis direction. The first member includes a tube body. Both ends of the tube body have an opening. An inner diameter of an edge of the opening of the tube body adjacent to the second member is smaller than the dimension of the limit portion in the direction perpendicular to the axis direction. The limit portion is disposed in the tube body. The tube body is integrally formed by stamping, stretching and blanking.

In some embodiments, a surface of the base is provided with the plating layer. The plating layer on the surface of the base is electrically connected to the plating layer on the surface of the columnar member. The base is integrally injection-molded with the plurality of columnar members.

In some embodiments, a conductive sheet is further included. The base has a connection hole configured to be connected to a connection member, to fix the conductive sheet to the substrate. The conductive sheet is electrically conductive with the plating layer on the surface of the base.

In some embodiments, the electroencephalography dry electrode further includes a second elastic member and a mounting member. The second elastic member is connected to a side of the base away from the columnar member. The second elastic member is extensible and retractable in an axial direction of the columnar member. The second elastic member is connected between the base and the mounting member.

In some embodiments, the electroencephalography dry electrode further includes a guide plate. The guide plate is disposed around an outer circumference of the second elastic member. At least one of the base and the mounting member is connected to the guide plate via a guide structure. The guide structure includes a guide groove and a guide portion movably inserted into the guide groove in the axial direction of the columnar member.

In a second aspect of the present disclosure, an electroencephalography measurement apparatus is further provided.

The electroencephalography measurement apparatus according to an embodiment of the second aspect of the present disclosure includes the electroencephalography dry electrode according to any one of the embodiments of the first aspect of the present disclosure.

In some embodiments, the apparatus includes a wearable assembly, a pushing assembly, and the electroencephalography dry electrode. The wearable assembly is adapted to be worn on a head of a user, and has a mounting cavity where the pushing assembly is located. The electroencephalography dry electrode is movably connected to the wearable assembly. The pushing assembly is configured to push the electroencephalography dry electrode to move towards the head, when a contact impedance between the electroencephalography dry electrode and the head is greater than or equal to a first impedance threshold.

In some embodiments, the pushing assembly is configured to push, at a reference pushing intensity, the electroencephalography dry electrode to move towards the head when the contact impedance between the electroencephalography dry electrode and the head is greater than or equal to the first impedance threshold. The reference pushing intensity is determined based on the contact impedance and a correspondence between an impedance and an intensity. The correspondence records a plurality of impedances, with any two of the plurality of impedances being different.

In some embodiments, the electroencephalography measurement apparatus further includes a main body, and a frontal lobe electrode group and a non-frontal lobe electrode group that are disposed at the main body. The frontal lobe electrode group includes a plurality of sheet-shaped dry electrodes mounted at a region of the main body corresponding to a frontal lobe brain region of a user. The non-frontal lobe electrode group includes a plurality of needle-shaped dry electrodes mounted at a region of the main body corresponding to a non-frontal lobe brain region of the user. The frontal lobe brain region includes at least one of a prefrontal lobe brain region and a postfrontal lobe brain region. The non-frontal lobe brain region includes at least one of a parietal lobe brain region, an occipital lobe brain region, and a temporal lobe brain region.

In some embodiments, at least two electrode group modules corresponding to different brain regions are provided. Each of the at least two electrode group modules has a rigid semi-ring structure, the at least two electrode group modules being connected with each other to form an integrated structure. At least one of the at least two electrode group modules has a retractable structure, enabling the module to move in a direction towards or away from a center of the module to adjust a dimension of the module. The brain regions include a prefrontal lobe brain region, a postfrontal lobe brain region, a parietal lobe brain region, an occipital lobe brain region and/or a temporal lobe brain region.

In some embodiments, the at least two electrode group modules include at least two of a first electrode group module corresponding to the prefrontal lobe brain region and/or the postfrontal lobe brain region, a second electrode group module corresponding to the occipital lobe brain region, and a third electrode group module corresponding to the parietal lobe brain region and/or the temporal lobe brain region.

In some embodiments, a first plug-in structure is formed at a connection part of the first electrode group module and the second electrode group module. At least one end of one module of the first electrode group module and the second electrode group module is formed with a protruding first plug-in block, and a corresponding end of the other module of the first electrode group module and the second electrode group module has a recessed first plug-in groove, the first plug-in block being configured to move in the first plug-in groove to achieve extension and retraction.

In some embodiments, the electroencephalography measurement apparatus further includes a main body, and a brain signal mainboard card, a plurality of electrodes, and a signal transmission line that are integrally disposed at the main body. The main body has an integrated rigid structure having a wiring channel formed inside or at a surface of the main body. The plurality of electrodes are grouped and arranged in at least one brain signal acquisition area of the main body. The wiring channel of the main body extends from the brain signal acquisition area to an area of the main body for mounting the brain signal mainboard card, and is configured to embed the signal transmission line. The signal transmission line is configured to connect the plurality of electrodes and the brain signal mainboard card, to transmit a brain signal acquired by the plurality of electrodes.

In some embodiments, the brain signal acquisition area of the main body includes one or more of a prefrontal lobe brain signal acquisition area, a postfrontal lobe brain signal acquisition area, a parietal lobe brain signal acquisition area, an occipital lobe brain signal acquisition area, and a temporal lobe brain signal acquisition area. The wiring channel includes a horizontal wiring channel and a vertical wiring channel, which are configured to guide the signal transmission line connected to the brain signal mainboard card to electrodes arranged at each brain signal acquisition area.

In some embodiments, the electrodes arranged in at least one of the brain signal acquisition areas includes an anode electrode and a cathode electrode used for transcranial direct current stimulation (TDCS).

In some embodiments, the wiring channel is embedded in the main body. The main body includes a first body, a second body, and a third body. An acquisition area of an occipital lobe brain region is located at an inner side of the second body, an acquisition area of a prefrontal lobe brain region and/or an acquisition area of a postfrontal lobe brain region are located at an inner side of the first body, and an acquisition area of a parietal lobe brain region and/or an acquisition area of a temporal lobe brain region are located at an inner side of the third body. The second body is fixedly arranged, with the first body being retractably plugged laterally in front of the second body, and the third body being retractably plugged longitudinally above the second body. The brain signal mainboard card is disposed in the second body, part of the horizontal wiring channel is formed in a plug-in structure of the first body, and part of the vertical wiring channel is formed in a plug-in structure of the third body.

Compared with the existing art, advantages of the electroencephalography measurement apparatus of the present disclosure are the same as those of the electroencephalography dry electrode of the embodiments of the present disclosure, and will not be repeated here.

Additional aspects and advantages of the present disclosure will be provided in part in the following description, or will in part become apparent from the following description or be learned from practicing of the present disclosure.

### BRIEF DESCRIPTION OF THE DRAWINGS

The above and/or additional aspects and advantages of the present disclosure will become more apparent and more understandable from the following description of embodiments taken in conjunction with the accompanying drawings.

FIG. 1 is a schematic structural view of an electroencephalography dry electrode according to an embodiment of the present disclosure.

FIG. 2 is a main view of an electroencephalography dry electrode according to an embodiment of the present disclosure.

FIG. 3 is a schematic structural view of a columnar member of an electroencephalography dry electrode according to an embodiment of the present disclosure.

FIG. 4 is a main view of a columnar member of an electroencephalography dry electrode according to an embodiment of the present disclosure.

FIG. 5 is a cross-sectional view of a columnar member of an electroencephalography dry electrode according to an embodiment of the present disclosure.

FIG. 6 is a first schematic structural view of an electroencephalography dry electrode according to another embodiment of the present disclosure, where an axial length of a columnar member is 8 mm.

FIG. 7 is a second schematic structural view of an electroencephalography dry electrode according to another embodiment of the present disclosure, where an axial length of a columnar member is 8 mm;

FIG. 8 is a first schematic structural view of an electroencephalography dry electrode according to another embodiment of the present disclosure, where an axial length of a columnar member is 10 mm.

FIG. 9 is a second schematic structural view of an electroencephalography dry electrode according to another embodiment of the present disclosure, where an axial length of a columnar member is 10 mm.

FIG. 10 is a first schematic structural view of an electroencephalography dry electrode according to yet another embodiment of the present disclosure, where an axial length of a columnar member is 8 mm.

FIG. 11 is a second schematic structural view of an electroencephalography dry electrode according to yet another embodiment of the present disclosure, where an axial length of a columnar member is 8 mm.

FIG. 12 is a first schematic structural view of an electroencephalography dry electrode according to yet another embodiment of the present disclosure, where an axial length of a columnar member is 10 mm.

FIG. 13 is a second schematic structural view of an electroencephalography dry electrode according to yet another embodiment of the present disclosure, where an axial length of a columnar member is 10 mm.

FIG. 14 is a schematic structural view of an electroencephalography dry electrode according to still another embodiment of the present disclosure.

FIG. 15 is a cross-sectional view of an electroencephalography measurement apparatus according to an embodiment of the present disclosure.

FIG. 16 is a partial schematic structural view of an electroencephalography measurement apparatus according to an embodiment of the present disclosure.

FIG. 17 is a schematic structural diagram of an electroencephalography measurement apparatus according to an embodiment of the present disclosure.

FIG. 18 is a schematic view of an electroencephalography measurement apparatus using a soft structure according to an embodiment of the present disclosure.

FIG. 19 is a top view of an electroencephalography measurement apparatus using a rigid structure according to another embodiment of the present disclosure.

FIG. 20 is a right view of an electroencephalography measurement apparatus using a rigid structure according to another embodiment of the present disclosure.

FIG. 21 is a perspective view of a retractable electroencephalography measurement apparatus according to an embodiment of the present disclosure.

FIG. 22 is a schematic structural view of a frontal lobe electrode group and a sheet-shaped dry electrode according to an embodiment of the present disclosure.

FIG. 23 is a schematic structural view of an occipital lobe electrode group (or a parietal lobe electrode group) and a needle-shaped dry electrode according to an embodiment of the present disclosure.

FIG. 24 is a schematic view of an electrode arrangement mode of a brain signal acquisition apparatus according to an embodiment of the present disclosure.

FIG. 25 is a human model database for a dimension design of a brain signal acquisition apparatus according to an embodiment of the present disclosure.

FIG. 26 is a schematic structural view of a pogo pin assembly used in a needle-shaped dry electrode according to an embodiment of the present disclosure.

FIG. 27 is a schematic structural view of a claw-shaped electrode used in a needle-shaped dry electrode according to another embodiment of the present disclosure.

FIG. 28 is a schematic structural view of a needle-shaped dry electrode using an active claw-shaped electrode according to another embodiment of the present disclosure.

FIG. 29 is a perspective view of an electroencephalography measurement apparatus according to an embodiment of the present disclosure.

FIG. 30 is a top view of an electroencephalography measurement apparatus according to an embodiment of the present disclosure.

FIG. 31 is a right view of an electroencephalography measurement apparatus according to an embodiment of the present disclosure.

FIG. 32 is an exploded view of an electroencephalography measurement apparatus according to an embodiment of the present disclosure.

FIG. 33 is a perspective view of an electroencephalography measurement apparatus after extension and retraction according to an embodiment of the present disclosure.

FIG. 34 is a partial enlarged view of FIG. 33.

FIG. 35 is a cross-sectional view of an electroencephalography measurement apparatus after extension and retraction according to an embodiment of the present disclosure.

FIG. 36 is a schematic structural view of an electroencephalography measurement apparatus according to an embodiment of the present disclosure, with part of a housing hidden.

FIG. 37 is a top view of an electroencephalography measurement apparatus according to an embodiment of the present disclosure.

FIG. 38 is a side view of an electroencephalography measurement apparatus according to an embodiment of the present disclosure.

FIG. 39 is a partial enlarged view of an electroencephalography measurement apparatus according to an embodiment of the present disclosure.

FIG. 40 is an exploded view of an electroencephalography measurement apparatus according to an embodiment of the present disclosure.

FIG. 41 is a schematic structural view of an electrode sheet according to an embodiment of the present disclosure.

FIG. 42 is a schematic structural view of an electrode needle according to an embodiment of the present disclosure.

FIG. 43 is a schematic view of a mounting structure of an electrode needle according to an embodiment of the present disclosure.

Reference numerals of the accompanying drawings:
electroencephalography dry electrode 100; base 10; connection hole 101; button male member 102; columnar member 20; first member 201; tube body 2011; cover body 2012; second member 202; recessed cavity 2021; limit portion 2022; first elastic member 203; conducive sheet 30; connection member 40; second elastic member 50; mounting member 60; guide plate 70; guide groove 801; guide portion 802; signal transmission line 90;
first semi-ring 1b; second semi-ring 2b; third semi-ring 3b; frontal lobe electrode group 4-1b; sheet-shaped dry electrode 41b; occipital lobe electrode group 4-2b; needle-shaped dry electrode 42b; pogo pin assembly 42A; ejector pin 42A-1; sleeve 42A-2; spring 42A-3; claw-shaped electrode 42B; base 42B-1; first electrode needle 42B-2; circuit board 43; parietal lobe electrode group 4-3b;
first electrode group module 1c; first plug-in block 11c; first limit groove 111c; second electrode group module 2c; first plug-in groove 21c; first locking tongue member 211c; first protruding end 2111c; second plug-in groove 22c; second limit groove 221c; three-way connection portion 25c; third electrode group module 3c; second plug-in block 31c; second locking tongue member 311c; second protruding end 3111c; guide post 32c; second electrode sheet 41c; second electrode needle 43c; second curved housing 51c; second electrode group support 52c; second electrode hole 521c; second intermediate layer 53c; function board 61c; second power board 62c; second ear clip 7c;
first body 1d; first locking structure 11d; slot 111d; second body 2d; third body 3d; second locking structure 31d; wire tube 32d; third electrode sheet 41d; third electrode needle 43d; third curved housing 51d; third electrode group support 52d; third electrode hole 521d; third intermediate layer 53d; brain signal mainboard card 61d; third power board 62d; third ear clip 7d; horizontal wiring channel 81d; vertical wiring channel 82d.

### DETAILED DESCRIPTION OF THE EMBODIMENTS

Embodiments of the present disclosure will be described in detail below with reference to examples thereof as illustrated in the accompanying drawings, throughout which same or similar elements, or elements having same or similar functions, are denoted by same or similar reference numerals. The embodiments described below with reference to the drawings are illustrative only, and are intended to explain, rather than limiting, the present disclosure.

In order to make objectives, technical solutions, and advantages of the embodiments of the present disclosure more clearly understood, technical solutions according to the embodiments of the present disclosure will be described clearly below in combination with accompanying drawings of the embodiments of the present disclosure. Obviously, the embodiments described below are only a part of the embodiments of the present disclosure, rather than all embodiments of the present disclosure. On a basis of the embodiments of the present disclosure, all other embodiments obtained by those skilled in the art without creative labor shall fall within the protection scope of the present disclosure.

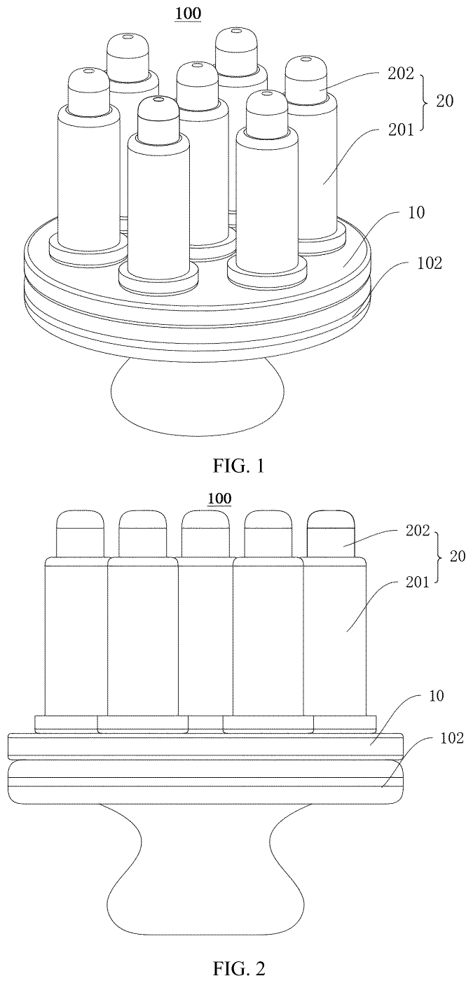

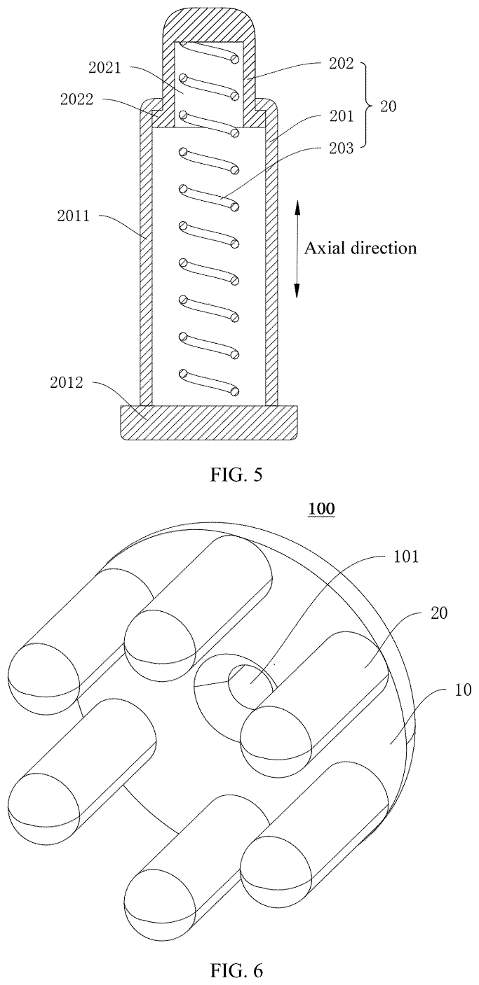

As illustrated in FIG. 1 to FIG. 14, an electroencephalography dry electrode 100 and an electroencephalography measurement apparatus according to the present disclosure are described below.

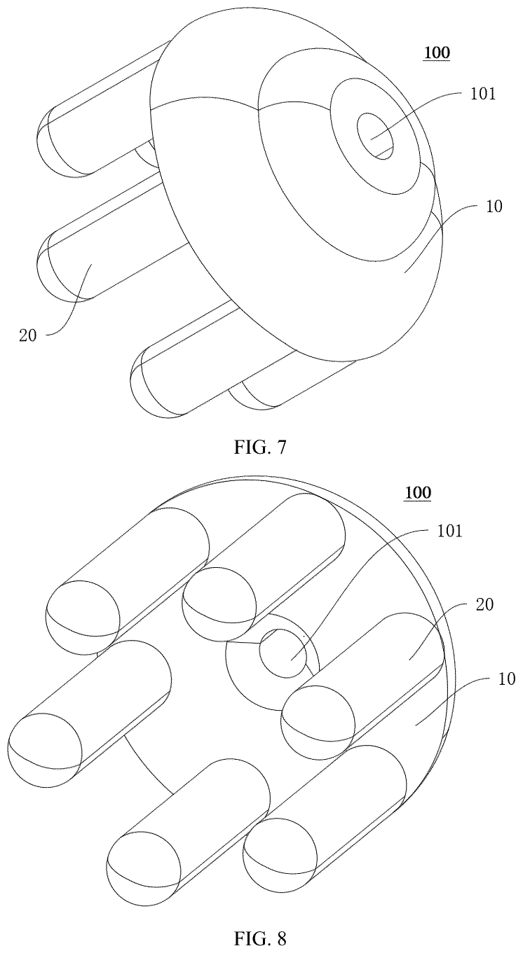

According to an embodiment of the present disclosure, the electroencephalography dry electrode 100 includes a substrate and a plating layer. The substrate includes a base 10 and a plurality of columnar members 20. An end of each of the plurality of columnar members 20 is fixed to the base 10 (as illustrated in FIG. 1, FIG. 2, and FIG. 6 to FIG. 14). At least a surface of each of the plurality of columnar members 20 is provided with the plating layer. The plating layer on the surface of each of the plurality of columnar members 20 is electrically connected to the base 10.

In an exemplary embodiment of the present disclosure, the substrate may be a copper member. Correspondingly, the plating layer may be a silver plating layer or a silver/silver chloride plating layer. In another exemplary embodiment of the present disclosure, the substrate may be an ABS material member (the ABS material is a terpolymer of acrylonitrile (A)-butadiene (B)-styrene(S)), and the plating layer may be a silver/silver chloride plating layer. In this way, first, a contact resistance between an electrode and a scalp is low. Second, the electrode has good reversibility and is not easily polarized. Even if a slightly large current flows through the electrode for a short period of time, a potential can quickly return to an initial state after power is turned off. Third, the potential of the electrode is relatively stable and close to zero potential, and is less likely to undergo polarization or passivation. Fourth, the potential has good reproducibility; a temperature coefficient is small, i.e., the potential changes little with a temperature; and after the temperature returns to an original temperature, the potential can quickly return to a potential at the original temperature. Fourth, preparation, actual use, and maintenance are relatively convenient and durable.

At least the surface of each of the plurality of columnar members 20 is provided with the plating layer. That is, the plating layer may be disposed only on the surface of the columnar member 20, and no plating layer may be disposed on a surface of the base 10. The base 10 includes a conductive metal material, and the plating layer on the surface of the columnar member 20 is electrically connected to the base 10. In another exemplary embodiment of the present disclosure, the plating layer may be disposed on both the surface of the columnar member 20 and the surface of the base 10, and the plating layer on the surface of the columnar member 20 is electrically connected to the plating layer on the surface of the base 10.

The substrate includes the base 10 and the plurality of columnar members 20. The end of each of the plurality of columnar members 20 is fixed to the base 10. It should be understood that, during use, the other end of each of the plurality of columnar members 20 contacts the scalp of a user, at least the surface of each of the plurality of columnar members 20 is provided with the plating layer, and the plating layer on the surface of each of the plurality of columnar members 20 is electrically connected to the base 10. In this way, an electroencephalographic signal can be transmitted to the base 10 via the plating layer on the surface of the columnar member 20, and the base 10 can be connected to a signal transmission line 90 to transmit the electroencephalographic signal to an external receiving device via the signal transmission line 90. Also, the base 10 can also be used for mechanical connection with an external fixing apparatus.

According to the electroencephalography dry electrode 100 of the embodiments of the present disclosure, the plurality of columnar members 20 are provided, and the end of each of the plurality of columnar members 20 is connected to the base 10. When it is needed to acquire the electroencephalography signal from a hairy area, the plurality of columnar members 20 pass through the hair, in such a manner that the other ends of the plurality of columnar members 20 contact the scalp. The plurality of columnar members 20 in the electroencephalography dry electrode 100 of the present disclosure can pass through the hair, in such a manner that the other ends of the plurality of columnar members 20 can maintain contact with the scalp, resolving a problem in the related art that a flat-plate dry electrode cannot be used in the hairy areas. Also, by providing the plurality of columnar members 20, a contact area between the electroencephalography dry electrode 100 and the scalp can be increased to reduce a contact impedance.

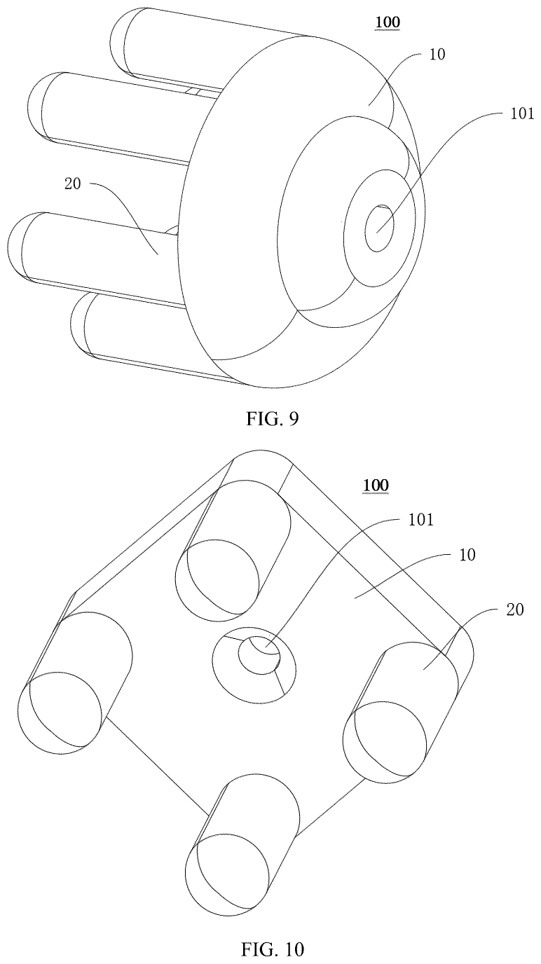

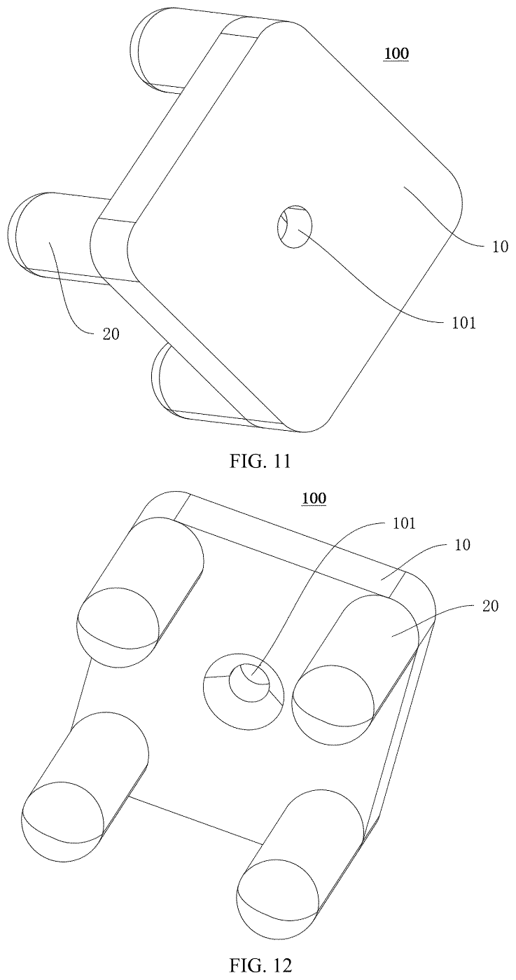

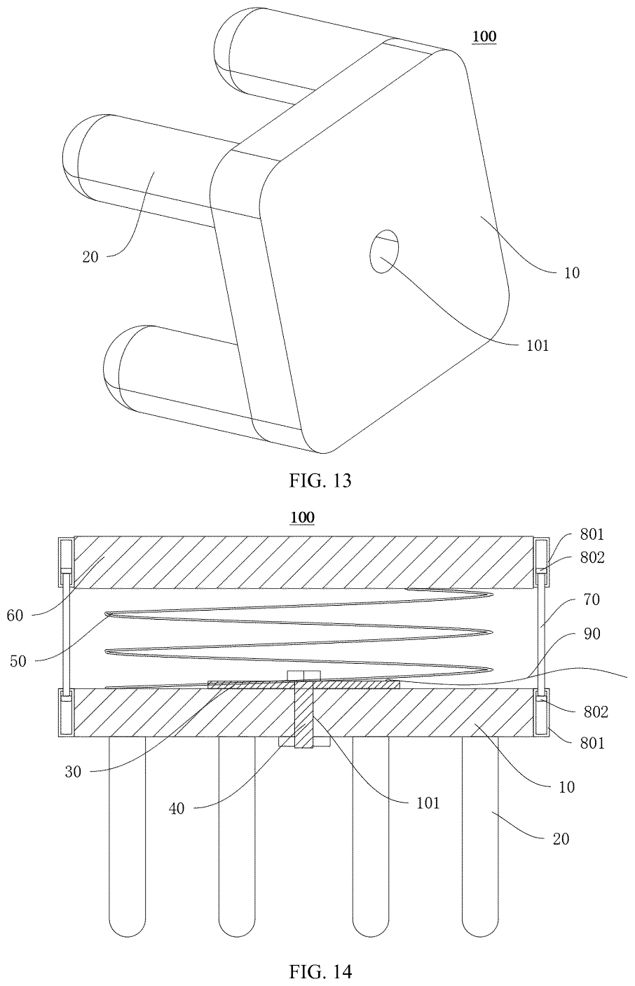

In some embodiments, a length of the columnar member 20 in an axial direction may range from 8 mm to 12 mm. For example, the length of the columnar member 20 in the axial direction may be 8 mm (for example, as illustrated in FIG. 6 and FIG. 7, FIG. 10 and FIG. 11), 9 mm, 10 mm (for example, as illustrated in FIG. 8 and FIG. 9, FIG. 12 and FIG. 13), 11 mm or 12 mm, or any value within a range between any two of the above length values. In this way, the columnar member 20 will not be excessive long, as an excessive length results in difficulty in fixing a position of the electroencephalography dry electrode 100; nor will the columnar member 20 be excessive short, and can be applied to hair of typical thickness, which is conducive to enable the electroencephalography dry electrode 100 to have good versatility.

In some embodiments, the number of the columnar members 20 may be reasonably determined based on a dimension of the base 10, different usage scenarios, etc. For example, the number of the columnar members 20 may be 3, 4 (as illustrated in FIG. 10 to FIG. 13), 5, 6 (as illustrated in FIG. 6 to FIG. 9), 7 (as illustrated in FIG. 1 and FIG. 2), 8, etc. For example, in an area with relatively sparse hair, an electroencephalography dry electrode 100 with a small number of columnar members 20 may be used, for example, an electroencephalography dry electrode 100 with four columnar members 20 (as illustrated in FIG. 10 to FIG. 13); and in an area with relatively dense hair, an electroencephalography dry electrode 100 with a large number of the columnar members 20 may be used, for example, an electroencephalography dry electrode 100 with six columnar members 20 (as illustrated in FIG. 6 to FIG. 9).

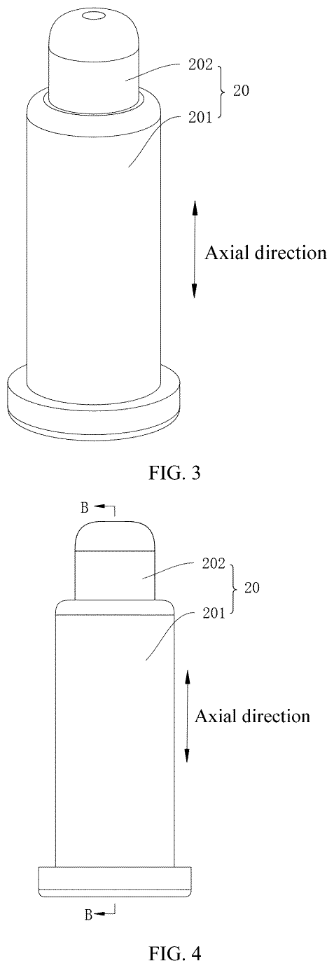

According to some embodiments of the present disclosure, as illustrated in FIG. 3 to FIG. 5, each of the plurality of columnar members 20 includes a first member 201, a second member 202, and a first elastic member 203. The first member 201 is sleeved at the second member 202. The second member 202 is configured to move relative to the first member 201 in an axial direction. The first elastic member 203 is disposed in the first member 201. Two ends of the first elastic member 203 are respectively connected to the first member 201and the second member 202. The first elastic member 203 is configured to apply a restoring force to the second member 202 to move outward from the first member 201. That is, when the second member 202 is pressed, the second member 202 will move inwardly towards the first member 201 in the axial direction. When an external force applied to the second member 202 stops, the second member 202 will move outwardly from the first member 201 in the axial direction under an action of the restoring force of the first elastic member 203. The axial direction is an axial direction of the columnar member 20.

To ensure that the other end of the columnar member 20 is in good contact with the scalp, when the columnar member 20 is a rigid columnar member, the columnar member 20 needs to be pressed tightly against the scalp of the user to ensure good contact between the columnar member 20 and the scalp. Wearing the columnar member 20 for a long time may cause discomfort to the user. In an embodiment of the present disclosure, the columnar member 20 is provided with the first member 201, the second member 202, and the first elastic member 203. Elastic properties of the columnar member 20 help ensure that the other end of the columnar member 20 can always form good contact with the scalp. Also, a pressing force on the scalp will not be excessive, which helps reduce the discomfort of the user.

In an exemplary embodiment of the present disclosure, the first elastic member 203 may be a spring. When the columnar member 20 including the first member 201, the second member 202 and the first elastic member 203 is extended or retracted, a minimum length of the columnar member 20 in the axial direction is not less than 8 mm.

According to some embodiments of the present disclosure, each of the plurality of columnar members 20 includes a copper member. Experimental studies have shown that when the columnar member 20 of the electroencephalography dry electrode 100 is the copper member, the potential of the electroencephalography dry electrode 100 is stable during use and does not change significantly as acquisition duration increases. As a result, acquired data has higher accuracy.

In some embodiments, as illustrated in FIG. 5, the first member 201 and the second member 202 are both the copper members. The second member 202 has a recessed cavity 2021. The recessed cavity 2021 has a first opening facing towards the first elastic member 203. The first elastic member 203 passes through the first opening with one end of the first elastic member 203 located in the recessed cavity 2021. The second member 202 is formed by stamping and stretching. The second member 202 has the recessed cavity 2021, and the recessed cavity 2021 has the first opening facing towards the first elastic member 203, in such a manner that the second member 202 can be easily formed by stamping and stretching. It should be noted that the copper member is relatively soft, and the second member 202 is an elongated member. Conventional milling results in a low yield rate for the second member 202, high processing difficulty, and a long processing time. An embodiment of the present disclosure utilizes a stamping and stretching method to manufacture the second member 202, which significantly reduces the processing difficulty, achieves a high yield rate, and achieves good structural accuracy for the second member 202. Also, a dimension of the columnar member 20 in the embodiments of the present disclosure is larger than a dimension of a conventional pogo pin, that is, a dimension of the second member 202 is relatively large, which is adapted to be integrally formed by stamping and stretching.

According to some embodiments of the present disclosure, the first member 201 and the second member 202 are both the copper members. As illustrated in FIG. 5, a limit portion 2022 is disposed at a circumferential edge of the first opening, and has a dimension greater than an outer diameter of the second member 202 in a direction perpendicular to an axis direction. The first member 201 includes a tube body 2011. Both ends of the tube body 2011 have an opening. An inner diameter of an edge of the opening of the tube body 2011 adjacent to the second member 202 is smaller than the dimension of the limit portion 2022 in the direction perpendicular to the axis direction. The limit portion 2022 is disposed in the tube body 2011. The tube body 2011 is integrally formed by stamping, stretching and blanking. It should be understood that, the limit portion 2022 is configured to cooperate with the edge of the opening of the tube body 2011 adjacent to the second member 202 to limit an extreme movement position of the second member 202 in the axial direction and in a direction away from the first member 201.

In an exemplary embodiment of the present disclosure, the tube body 2011 is integrally formed by stamping, stretching and blanking, which includes: stamping and stretching a copper plate to enable the cooper plate to form a first intermediate member having a recessed portion, one end open, and the other end closed; then blanking the other end surface of the first intermediate member to enable the first intermediate member to form an opening at the other end surface of the first intermediate member, thereby obtaining a second intermediate member. A diameter of an opening at the other end surface of the second intermediate member is smaller than an inner diameter of a circumferential side wall of the second intermediate member, and the second intermediate member may serve as the first member 201. Alternatively, a blanking operation may be performed on an edge of an opening at one end of the second intermediate member to remove a portion of the edge of the opening at one end of the second intermediate member that protrudes from the circumferential side wall of the second intermediate member in the direction perpendicular to the axis direction, thereby obtaining the first member 201.

It should be noted that the copper member is relatively soft, and the tube body 2011 is an elongated member. The conventional milling results in a low yield rate for the tube body 2011, high processing difficulty, and a long processing time. An embodiment of the present disclosure utilizes an integrally stamping, stretching, and blanking method to manufacture the tube body 2011, significantly reducing the processing difficulty of the tube body 2011, achieving a high yield rate, and achieving good structural accuracy for the tube body 2011. Also, the dimension of the columnar member 20 in the embodiments of the present disclosure is larger than the dimension of the conventional pogo pin, that is, a dimension of the tube body 2011 is relatively large, which is adapted to be integrally formed by stamping, stretching, and blanking.

According to some embodiments of the present disclosure, the first member 201 further includes a cover body 2012 covering an opening of the tube body 2011 away from the second member 202. The first elastic member 203 is connected to the cover body 2012. In an exemplary embodiment of the present disclosure, the cover body 2012 is a flat plate member. During assembly, the second member 202 may be placed inside the first member 201 first, then the first elastic member 203 may be placed inside the first member 201, and finally the cover body 2012 covers the opening of the tube body 2011 away from the second member 202. In another exemplary embodiment of the present disclosure, the second member 202 may be placed inside the first member 201 first, the first elastic member 203 may be connected to the cover body 2012, then the first elastic member 203 may be placed inside the first member 201, and the cover body 2012 covers the opening of the tube body 2011 away from the second member 202. The cover body 2012 may be connected to the tube body 2011 by welding.

The first member 201 includes the tube body 2011 and the cover body 2012. In this way, the tube body 2011 is facilitated to be manufactured by integrally stamping, stretching, and blanking, which is conducive to reducing a manufacturing difficulty of the first member 201.

According to some embodiments of the present disclosure, the surface of the base 10 is provided with the plating layer. The plating layer on the surface of the base 10 is electrically connected to the plating layer on the surface of the columnar member 20. The base 10 is integrally injection-molded with the plurality of columnar members 20. That is, the columnar member 20 and the base 10 may form an integrated member. The base 10 and the columnar member 20 are manufactured by an integrated injection molding process. On the one hand, continuity of the plating layer on the surface of the base 10 and the plating layer on the surface of the columnar member 20 are better; on the other hand, the plating layer has better bonding performance with the surface of the base 10 and the surface of the columnar member 20, and the plating layer is less likely to fall off.

According to some embodiments of the present disclosure, the electroencephalography dry electrode 100 further includes a conductive sheet 30. The base 10 has a connection hole 101 configured to be connected to a connection member 40, to fix the conductive sheet 30 to the substrate. The conductive sheet 30 is electrically conductive with the plating layer on the surface of the base 10. It should be noted that, since the base 10 and the plurality of the columnar members 20 are injection-molded members, direct welding of the signal transmission line 90 to the surface of the base 10 is not possible. In an embodiment of the present disclosure, the conductive sheet 30 is fixed at the base 10, one end of the signal transmission line 90 may be welded to the conductive sheet 30, and the conductive sheet 30 is electrically connected to the plating layer on the surface of the base 10. In this way, an electrical signal can be transmitted to the external receiving device through the plating layer on the surface of the base 10, the conductive sheet 30, and the signal transmission line 90.

In some embodiments, one end of the signal transmission line 90 may be clamped between the conductive sheet 30 and the plating layer on the surface of the base 10, in such a manner that the signal transmission line 90 may also be electrically connected to the plating layer on the surface of the base 10.

In some embodiments, the conductive sheet 30 may be disposed at a side of the base 10 where the columnar member 20 is disposed, or the conductive sheet 30 may also be disposed at a side of the base 10 facing away from the columnar member 20. The conductive sheet 30 is disposed at the side of the base 10 facing away from the columnar member 20, and one end of the signal transmission line 90 is connected to a side of the conductive sheet 30 facing away from the base. In this way, on the one hand, the signal transmission line 90 can be kept as far away from the hair as possible; on the other hand, the conductive sheet 30 and one end of the signal transmission line 90 can be prevented from occupying a space defined among the plurality of columnar members 20. As a result, a length of the columnar member 20 can be facilitated to be as short as possible.

In an exemplary embodiment of the present disclosure, as illustrated in FIG. 14, the connection member 40 may be a screw and a nut. During connection, the screw penetrates the conductive sheet 30 and the connection hole 101, and the nut is then tightened onto the screw to complete fixation. The connection is convenient and fast, with good connection stability. In another exemplary embodiment of the present disclosure, the connection member 40 may be an adhesive member, with one end of the adhesive member bonded to the side of the conductive sheet 30 facing away from the base 10 and the other end of the adhesive member bonded to the base 10 to fix the conductive sheet 30. In another exemplary embodiment of the present disclosure, the connection member 40 may be a screw. The screw penetrates the conductive sheet 30 and extends into the connection hole 101, and is directly threadedly connected to the connection hole 101.

According to some embodiments of the present disclosure, the base 10 and the plurality of columnar members 20 include an ABS material member (the ABS material is the terpolymer of acrylonitrile (A)-butadiene (B)-styrene(S)). Experimental studies have shown that the electroencephalography dry electrode 100 whose the columnar member 20 is the ABS material member has a stable potential during use and does not change significantly as the acquisition duration increases, leading to highly accurate data.

In some embodiments, the ABS material member is a rigid ABS material member.

According to some embodiments of the present disclosure, as illustrated in FIG. 14, the electroencephalography dry electrode 100 further includes a second elastic member 50 and a mounting member 60. The second elastic member 50 is connected to a side of the base 10 away from the columnar member 20. The second elastic member 50 is extensible and retractable in the axial direction of the columnar member 20. The second elastic member 50 is connected between the base 10 and the mounting member 60. To ensure that the other end of the columnar member 20 is in good contact with the scalp, when the columnar member 20 is the rigid columnar member, the columnar member 20 needs to be pressed tightly against the scalp of the user to ensure the good contact between the columnar member 20 and the scalp. However, wearing the columnar member 20 for a long time may cause discomfort to the user. In an embodiment of the present disclosure, the second elastic member 50 is disposed at the side of the base 10 facing away from the columnar member 20, in such a manner that the second elastic member 50 helps ensure that the other end of the columnar member 20 can always form good contact with the scalp. Also, the pressing force on the scalp will not be excessive, which helps reduce the discomfort of the user. The mounting member 60 is configured to be connected to the external fixing apparatus. In an exemplary embodiment of the present disclosure, the second elastic member 50 may be a spring.

According to some embodiments of the present disclosure, the electroencephalography dry electrode 100 further includes a flexible shielding member, which is disposed around an outer circumference of the second elastic member 50. Edges of the flexible shielding member at both ends in the axial direction are connected to the base 10 and the mounting member 60, respectively. In an exemplary embodiment of the present disclosure, the flexible shielding member may be a silicone member, a rubber member, a leather layer, a cloth, or the like.

According to some embodiments of the present disclosure, as illustrated in FIG. 14, the electroencephalography dry electrode 100 further includes a guide plate 70. The guide plate 70 is disposed around the outer circumference of the second elastic member 50. At least one of the base 10 and the mounting member 60 is connected to the guide plate 70 via a guide structure. The guide structure includes a guide groove 801 and a guide portion 802 movably inserted into the guide groove 801 in the axial direction of the columnar member 20. It should be understood that, on the one hand, the guide plate 70 cooperates with the guide structure to guide relative movement between the base 10 and the mounting member 60; on the other hand, the guide plate 70 can shield the second elastic member 50, which is conducive to reducing the possibility of the second elastic member 50 contacting the hair, further reducing the possibility of the hair and the second elastic member 50 being entangled; on the third hand, an appearance of the electroencephalography dry electrode 100 is also more aesthetically pleasing; additionally, connection stability between the base 10 and the mounting member 60 can be improved, in such a manner that the base 10 and the mounting member 60 will not be staggered in the direction perpendicular to the axis direction.

In an exemplary embodiment of the present disclosure, the guide plate 70 may be a ring-shaped plate member disposed around the outer circumference of the second elastic member 50 (as illustrated in FIG. 14). In another exemplary embodiment of the present disclosure, a plurality of guide plates 70 are provided at intervals in a circumferential direction of the second elastic member 50, and the plurality of guide plates 70 are disposed around the outer circumference of the second elastic member 50.

In some embodiments, one guide structure is provided. The guide structure is disposed at the base 10, that is, the guide groove 801 is disposed at the base 10, the guide plate 70 is connected between the mounting member 60 and the guide portion 802, and the guide portion 802 is movably inserted into the guide groove 801 in the axial direction of the columnar member 20.

In some embodiments, one guide structure is provided. The guide structure is disposed at the mounting member 60, that is, the guide groove 801 is disposed at the mounting member 60, the guide plate 70 is connected between the base 10 and the guide portion 802, and the guide portion 802 is movably inserted into the guide groove 801 in the axial direction of the columnar member 20.

As illustrated in FIG. 12, in some embodiments, two guide structures are provided. The two guide structures are respectively disposed on the base 10 and the mounting member 60, that is, two guide grooves 801 are respectively disposed at the base 10 and the mounting member 60, two guide portions 802 are respectively connected to two ends of the guide plate 70 in the axial direction, and the guide portions 802 are movably inserted into the guide grooves 801 in the axial direction of the columnar member 20. In this way, a range of the relative movement between the base 10 and the mounting member 60 is larger.

In some embodiments, as illustrated in FIG. 1 and FIG. 3, the base 10 may be a circuit board, and the electroencephalography dry electrode 100 further includes a button male member 102. The base 10 may be connected to the button male member 102, for example, by welding. The external fixing apparatus includes a button female member. The button male member 102 and the button female member may be plugged into each other for quick connection.

The present disclosure further provides an electroencephalography measurement apparatus.

According to an embodiment of the present disclosure, the electroencephalography measurement apparatus includes the electroencephalography dry electrode 100 according to any one of the embodiments of the present disclosure.

Compared with the existing art, advantages of the electroencephalography measurement apparatus of the present disclosure are the same as those of the electroencephalography dry electrode 100 of the embodiments of the present disclosure, and will not be repeated here.

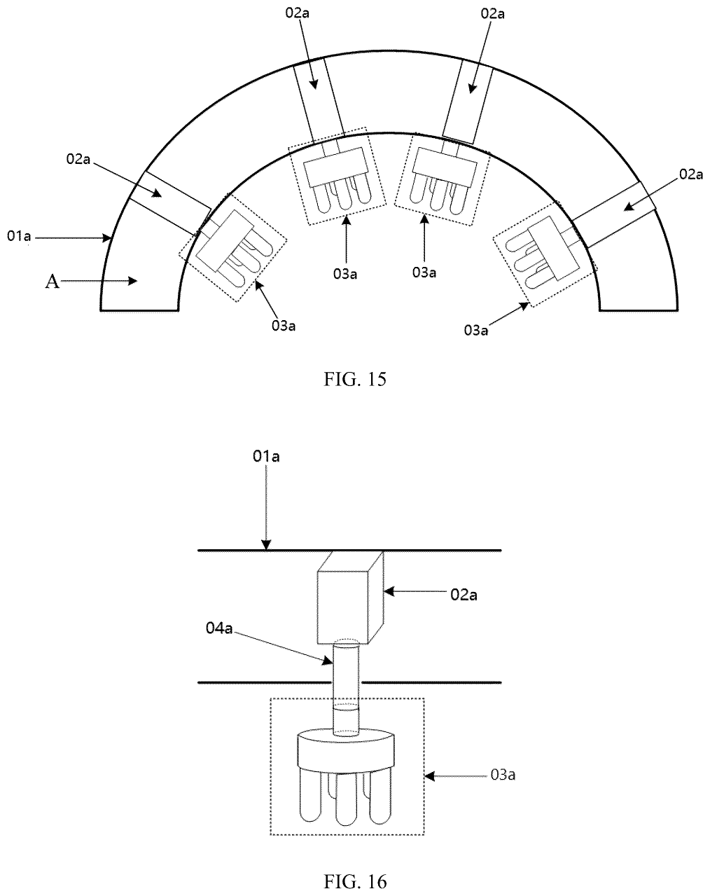

In some embodiments of the present disclosure, the electroencephalography measurement apparatus is provided. As illustrated in FIG. 15, the apparatus includes a wearable assembly 01a, a pushing assembly 02a, and a signal acquisition electrode 03a. As can be seen from FIG. 15, the apparatus may include a plurality of pushing assemblies 02a and a plurality of signal acquisition electrodes 03a. The plurality of signal acquisition electrodes 03a are in one-to-one correspondence with the plurality of pushing assemblies 02a.

The wearable assembly Ola is adapted to be worn on a head of the user, and has a mounting cavity A where the pushing assembly 02a is located. The signal acquisition electrode 03a is movably connected to the wearable assembly 01a. For example, the signal acquisition electrode 03a may be movably connected to an inner side of the wearable assembly 01a (i.e., a side adjacent to the head of the user).

After the user wears the wearable assembly Ola on the head of the user, the pushing assembly 02a is configured to push the signal acquisition electrode 03a to move towards the head, when a contact impedance between the signal acquisition electrode 03a and the head is greater than or equal to a first impedance threshold. In this way, the contact impedance between the signal acquisition electrode 03a and the head is automatically adjusted. The contact impedance between the signal acquisition electrode 03a and the head refers to a contact impedance between the signal acquisition electrode 03a and skin of the head.

It should be understood that, the pushing assembly 02a may push the signal acquisition electrode 03a for a plurality of times until the contact impedance is less than the first impedance threshold. The first impedance threshold refers to a maximum contact impedance allowed between the signal acquisition electrode 03a and the head of the user when signal quality of an electroencephalography signal acquired by the electroencephalography measurement apparatus meets quality requirements. In other words, the first impedance threshold is the maximum contact impedance that does not affect quality of electroencephalography signal acquisition.

In summary, the embodiments of the present disclosure provide an electroencephalography measurement apparatus. The apparatus includes a wearable assembly, a signal acquisition electrode movably connected to the wearable assembly, and the pushing assembly located in the mounting cavity of the wearable assembly. The pushing assembly is configured to automatically push the signal acquisition electrode to move towards the head of the user when the contact impedance between the signal acquisition electrode and the head of the user is greater than or equal to the first impedance threshold, in such a manner that the signal acquisition electrode is in full contact with the head. Since the staff does not need to manually adjust the signal acquisition electrode to make the signal acquisition electrode fully come into contact with the head, the electroencephalography measurement apparatus provided in the embodiments of the present disclosure has a high degree of intelligence and can simplify operations of the staff.

In an embodiment of the present disclosure, as illustrated in FIG. 16, the electroencephalography measurement apparatus may further include a conductive rod 04a. The mounting cavity A of the wearable assembly Ola has a through hole at a side of the mounting cavity A (not illustrated in FIG. 16). The conductive rod 04a may be located inside the through hole to be movably connected to the wearable assembly 01a. The conductive rod 04a has an end connected to the signal acquisition electrode 03a, and another end abutting with the pushing assembly 02a.

The pushing assembly 02a is configured to push the conductive rod 04a to move towards the head of the user, when the contact impedance between the signal acquisition electrode 03a and the head of the user is greater than or equal to the first impedance threshold. The conductive rod 04a can then drive the signal acquisition electrode 03a to move towards the head of the user.

In an embodiment of the present disclosure, the electroencephalography measurement apparatus may be connected to an electronic device (such as a computer). After the user wears the wearable assembly 01a on the head of the user, the electronic device may obtain the contact impedance between the signal acquisition electrode 03a and the head, and may control the pushing assembly 02a to automatically push the signal acquisition electrode 03a to move towards the head when it is determined that the contact impedance is greater than or equal to the first impedance threshold. The electronic device may be independent of the electroencephalography measurement apparatus, and the electronic device may pre-store the first impedance threshold.

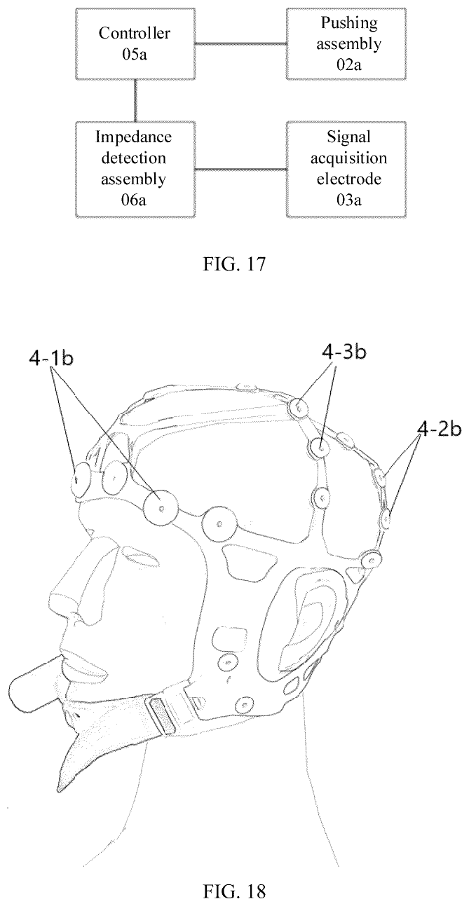

In another exemplary embodiment of the present disclosure, as illustrated in FIG. 17, the electroencephalography measurement apparatus may further include a controller 05a. The controller 05a may be connected to the pushing assembly 02a. After the user wears the wearable assembly Ola on the head of the user, the controller 05a may obtain the contact impedance between the signal acquisition electrode 03a and the head. If the controller 05a determines that the contact impedance is greater than or equal to the first impedance threshold, it can be determined that the signal acquisition electrode 03a is not in full contact with the skin of the head. Then, the controller 05a may control the pushing assembly 02a to push the signal acquisition electrode 03a to move towards the head, in such a manner that the signal acquisition electrode 03a can be in full contact with the head. The controller 05a may pre-store the first impedance threshold.

In the embodiments of the present disclosure, there are many ways to implement the pushing assembly 02a. The embodiments of the present disclosure take the following optional implementations as examples to illustrate the pushing assembly 02a.

In a first optional implementation, the pushing assembly 02a may be an air bladder. The air bladder has an end that may be fixedly connected to an inner wall of the mounting cavity A of the wearable assembly Ola, such as by adhesion. The air bladder has another end that may abut with the signal acquisition electrode 03a after inflation. The air bladder is configured to inflate when the contact impedance between the signal acquisition electrode 03a and the head is greater than or equal to the first impedance threshold, to push the signal acquisition electrode 03a to move towards the head.

For the first implementation, the electroencephalography measurement apparatus may further include an inflation assembly connected to the air bladder. The inflation assembly may be configured to inflate the air bladder when the contact impedance between the signal acquisition electrode 03a and the head is greater than or equal to the first impedance threshold. In another exemplary embodiment of the present disclosure, the inflation assembly may be a blower or an air pump.

In an embodiment of the present disclosure, the electroencephalography measurement apparatus may further include an air tube. The air tube has an end that may be connected to the air bladder, and another end that may be connected to the inflation assembly. In this way, the inflation assembly may inflate the air bladder through the air tube.

It should be understood that, the inflation assembly may be connected to the controller 05a of the electroencephalography measurement apparatus. The controller 05a is configured to control the inflation assembly to inflate the air bladder when the contact impedance between the signal acquisition electrode 03a and the head is greater than or equal to the first impedance threshold. In another exemplary embodiment of the present disclosure, the inflation assembly may be connected to the electronic device. The electronic device is configured to control the inflation assembly to inflate the air bladder when the contact impedance between the signal acquisition electrode 03a and the head is greater than or equal to the first impedance threshold.

It should be understood that, the mounting cavity A of the wearable assembly 01a may have a receiving groove at an inner wall of the mounting cavity A. The air bladder may be disposed in the receiving groove, for example, may be bonded to a groove wall of receiving groove. The air bladder may be located in the receiving groove when not inflated, to realize storage of the air bladder. After the air bladder is inflated, part of the air bladder may be located outside the receiving groove.

In a second optional implementation, the pushing assembly 02a may be a retractable assembly, such as a retractable rod. The retractable assembly has an end that may be connected to the inner wall of the mounting cavity A of the wearable assembly 01a, and another end that may abut with the signal acquisition electrode 03a after being in an extended state. The retractable assembly is configured to be in the extended state when the contact impedance between the signal acquisition electrode and the head is greater than or equal to the first impedance threshold, to push the signal acquisition electrode 03a to move towards the head of the user.

In a third optional implementation, the pushing assembly 02a may be an electromagnet connected to the signal acquisition electrode 03a. In this case, the inner wall of the mounting cavity A of the wearable assembly 01a may further be provided with a magnet. The magnet is arranged relative to the pushing assembly 02a. For example, an orthographic projection of the magnet on a plane where the pushing assembly 02a is located overlaps with the pushing assembly 02a, or is located in the pushing assembly 02a. The magnet may be a permanent magnet or an electromagnet.

The pushing assembly 02a is configured to be energized when the contact impedance between the signal acquisition electrode and the head is greater than or equal to the first impedance threshold, to generate a magnetic field opposite to that of the magnet. Thus, the signal acquisition electrode 03a is pushed to move towards the head of the user.

For the third optional implementation, the electroencephalography measurement apparatus may further include a power supply assembly. The power supply assembly may be connected to the pushing assembly 02a and may be configured to provide an electrical signal for the pushing assembly 02a. In this way, the pushing assembly 02a may generate a magnetic field opposite to that of the mounting member.

For example, the power supply assembly may provide the electrical signal for the pushing assembly 02a under control of the controller 05a of the electroencephalography measurement apparatus or control of the electronic device. The electrical signal may be a current signal or a voltage signal.

In an embodiment of the present disclosure, as illustrated in FIG. 17, the electroencephalography measurement apparatus may further include an impedance detection assembly 06a. As illustrated in FIG. 17, the impedance detection assembly 06a may be connected to the signal acquisition electrode 03a and the controller 05a. When the electroencephalography measurement apparatus is worn on the head of the user and an impedance test process is initiated, the impedance detection assembly 06a detects the contact impedance between the signal acquisition electrode 03a and the head of the user, and transmits the contact impedance to the controller 05a. Accordingly, the controller 05a obtains the contact impedance.

In another exemplary embodiment of the present disclosure, the pushing assembly 02a is configured to push, at a reference intensity corresponding to the contact impedance between the signal acquisition electrode 03a and the head, the signal acquisition electrode 03a. The reference intensity is determined based on the contact impedance and a correspondence between an impedance and an intensity. The correspondence records a plurality of impedances, with any two of the plurality of impedances being different. Any two intensities are also different.

As can be seen, the pushing assembly 02a may push the signal acquisition electrode 03a at different intensities. In this way, control flexibility of the signal acquisition electrode 03a is improved.

It should be understood that, the intensity recorded in the correspondence may increase as the impedance increases, that is, the intensity recorded in the correspondence is positively correlated with the impedance. In this way, when the contact impedance is large, the signal acquisition electrode may be pushed with a large pushing intensity. Therefore, the signal acquisition electrode 03a is allowed to be in contacted with the head quickly and closely, shortening the time taken for the contact impedance between the signal acquisition electrode and the head to drop below the first impedance threshold, and improving the control efficiency of the signal acquisition electrode.

In an embodiment of the present disclosure, the controller 05a (or the electronic device) may pre-store the correspondence between the impedance and the intensity. After the controller 05a (or the electronic device) obtains the contact impedance between the signal acquisition electrode and the head of the user, a reference pushing intensity corresponding to the contact impedance may be determined based on the correspondence.

It should be understood that, if the pushing assembly 02a is an air bladder, the intensity recorded in the correspondence between the impedance and the intensity may refer to an inflation volume of the inflation assembly for the air bladder each time. If the pushing assembly 02a is a retractable assembly, the intensity recorded in the correspondence between the impedance and the intensity may refer to an extension length of the retractable assembly each time. If the pushing assembly 02a is an electromagnet, the intensity recorded in the correspondence between the impedance and the intensity may refer to a magnitude of an electrical signal provided by the power supply assembly for the electromagnet each time. In an embodiment of the present disclosure, if the controller 05a (or the electronic device) determines that contact impedances of a target number of signal acquisition electrodes 03a among the plurality of signal acquisition electrodes 03a of the electroencephalography measurement apparatus meet a target condition, then the electroencephalography signal may be acquired using the plurality of signal acquisition electrodes 03a. The target number is less than or equal to a total number of the signal acquisition electrodes 03a, and is greater than a threshold number. The target condition is that a duration during which the contact impedance is less than the first impedance threshold reaches a target duration. Both the threshold number and the target duration may be pre-stored by the controller 05a. For example, the threshold number may be four-fifths of the total number. The target duration may be 10 minutes.

For example, the target number may be the total number of the signal acquisition electrodes. That is, after the controller 05a (or electronic device) determines that the contact impedances of all the signal acquisition electrodes 03a of the electroencephalography measurement apparatus meet the target condition, the electroencephalography signal of the user may be acquired. In other words, the controller 05a only begins acquiring the electroencephalography signal after determining that the signal acquisition electrode 03a is relatively stable relative to the head of the user. In this way, presence of excessive noise in the acquired electroencephalography signal can be avoided, ensuring good quality of the acquired electroencephalography signal.

According to the above description, the electroencephalography measurement apparatus provided in the embodiments of the present disclosure can realize automatic adjustment of the contact impedance, and can automatically start acquiring the electroencephalography signal after the contact impedances of the target number of the signal acquisition electrodes 03a all meet the target condition.

In an embodiment of the present disclosure, the signal acquisition electrode 03a may be a needle-shaped electrode. The wearable assembly 01a may be cap-shaped, or the wearable assembly 02 may be ring-shaped. In a case where the wearable assembly 01a is ring-shaped, the electroencephalography measurement apparatus may further include a plurality of sheet-shaped electrodes. The plurality of sheet-shaped electrodes and the plurality of needle-shaped electrodes may be arranged opposite to each other. For example, the number of the sheet-shaped electrodes may be five and the number of the needle-shaped electrodes may be four. One of the five sheet-shaped electrodes serves as a ground (GND) electrode. That is, the electroencephalography measurement apparatus is an 8-channel electroencephalography measurement apparatus.

In a process of acquiring the electroencephalography signal of the user, the plurality of sheet-shaped electrodes may be in contact with a forehead of the user, and the plurality of needle-shaped electrodes may be in contact with a back of the head of the user.

In another exemplary embodiment of the present disclosure, the wearable assembly 01a may be made of a flexible material.

In summary, the embodiments of the present disclosure provide the electroencephalography measurement apparatus. The apparatus includes the wearable assembly, the signal acquisition electrode movably connected to the wearable assembly, and the pushing assembly located in the mounting cavity of the wearable assembly. The pushing assembly is configured to automatically push the signal acquisition electrode to move towards the head of the user when the contact impedance between the signal acquisition electrode and the head of the user is greater than or equal to the first impedance threshold, in such a manner that the signal acquisition electrode is in full contact with the head. Since the staff does not need to manually adjust the signal acquisition electrode to make the signal acquisition electrode fully come into contact with the head, the electroencephalography measurement apparatus provided in the embodiments of the present disclosure has the high degree of intelligence and can simplify the operations of the staff.

In view of the drawback of poor brain signal acquisition performance in the existing art that uses a brain signal acquisition apparatus with a single form, in some embodiments of the present disclosure, an electroencephalography measurement apparatus is provided. This electroencephalography measurement apparatus adopts dual-form electrodes for different acquisition regions, which reduces a difficulty of brain signal acquisition and improves signal quality of the brain signal acquisition.

In some embodiments of the present disclosure, the electroencephalography measurement apparatus is provided. The acquisition apparatus includes a main body, and a frontal lobe electrode group 4-1b and a non-frontal lobe electrode group that are disposed at the main body. The frontal lobe electrode group 4-1b includes a plurality of sheet-shaped dry electrodes 41b mounted at a region of the main body corresponding to a frontal lobe brain region of the user. The non-frontal lobe electrode group includes a plurality of needle-shaped dry electrodes 42b mounted at a region of the main body corresponding to a non-frontal lobe brain region of the user. The frontal lobe brain region includes at least one of a prefrontal lobe brain region and a postfrontal lobe brain region. The non-frontal lobe brain region includes at least one of a parietal lobe brain region, an occipital lobe brain region, and a temporal lobe brain region.

A frontal lobe, a parietal lobe, an occipital lobe, and a temporal lobe are common EEG signal acquisition regions.

Regarding the frontal lobe brain region, the frontal lobe is located in an anterior part of the brain, and includes a prefrontal lobe and a postfrontal lobe. As an anterior part of the frontal lobe, the prefrontal lobe is adjacent to an inferior margin of a frontal bone and is involved in functions such as emotion, decision-making, cognitive control, and social behavior. As a posterior part of the frontal lobe, the postfrontal lobe is involved in functions such as visual information processing and spatial cognition. As for the non-frontal lobe brain region, the parietal lobe is located in a central area of the brain and is involved in functions such as sensory information processing and attention. The occipital lobe is located in the posterior part of the brain and is involved in functions such as visual information processing and spatial navigation. The temporal lobe is located at a side of the brain and is involved in functions such as auditory information processing, language, and memory.

Based on contact characteristics of different regions of the head, the electroencephalography measurement apparatus provided in the embodiments of the present disclosure introduces a dual-form dry electrode. The sheet-shaped dry electrode 41b is arranged in a region with few or no hair (such as the frontal lobe brain region or its prefrontal lobe brain region) to acquire a brain signal in the region. Arrangement of sheet-shaped dry electrode 41b allows for more direct contact with the scalp, improving stability and sensitivity of the signal, and effectively acquiring the brain signal from the region. The needle-shaped dry electrode 42b is located in a region with dense hair (such as the parietal lobe brain region, the occipital lobe brain region, and temporal lobe brain region) to acquire the brain signal from the region. Use of the needle-shaped dry electrode 42b can penetrate the hair and more easily contact the scalp, reducing hair interference with signal contact. Also, contact stability between the electrode and the scalp can be better maintained, which helps to improve the signal quality of the brain signal acquisition in the brain region.

Compared with the frontal lobe brain region which still uses the needle-shaped dry electrode for acquisition, the acquisition apparatus provided in the embodiments of the present disclosure uses the sheet-shaped dry electrode in the frontal lobe brain region, which is more comfortable for a subject to wear. The sheet-shaped dry electrode is more firmly fixed, and the signal acquisition is more stable.

In the above embodiments, arrangement of the dual-form dry electrode fully accounts for the characteristics of different regions of the head, effectively reducing the difficulty of the brain signal acquisition, and improving the signal quality of the acquired brain signal. This design not only improves accuracy and reliability of the brain signal acquisition, but also enhances comfort and engagement of the subject, better meeting needs of scientific research and clinical applications.

An advantage of the brain signal acquisition apparatus is that the brain signal acquisition apparatus can be flexibly adjusted based on the characteristics of different regions of the head, reducing the difficulty of the brain signal acquisition, and improving the quality and the reliability of the signal. This design is expected to be widely used in the field of brain signal acquisition, providing more reliable and accurate brain signal data for the scientific research, medical treatment and other fields, and helping to promote the development and progress of related fields.

In some embodiments, the body has an integrated rigid structure or an integrated elastic cap structure.

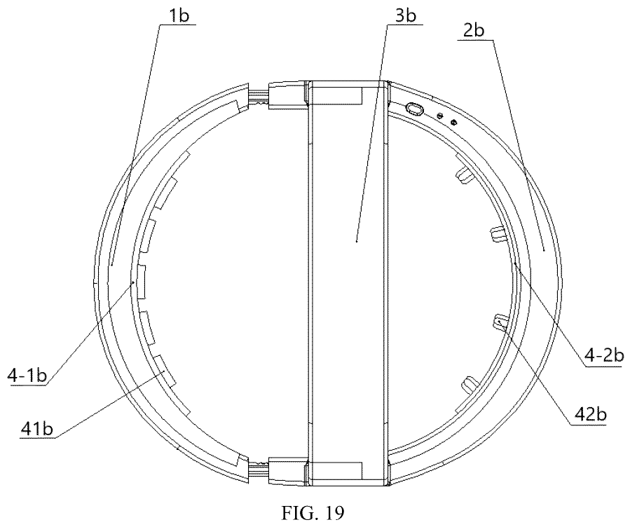

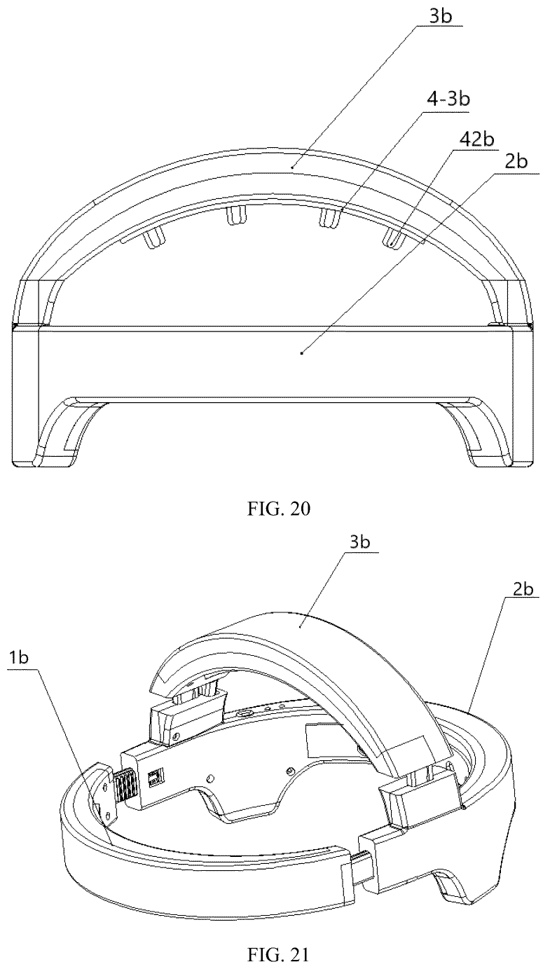

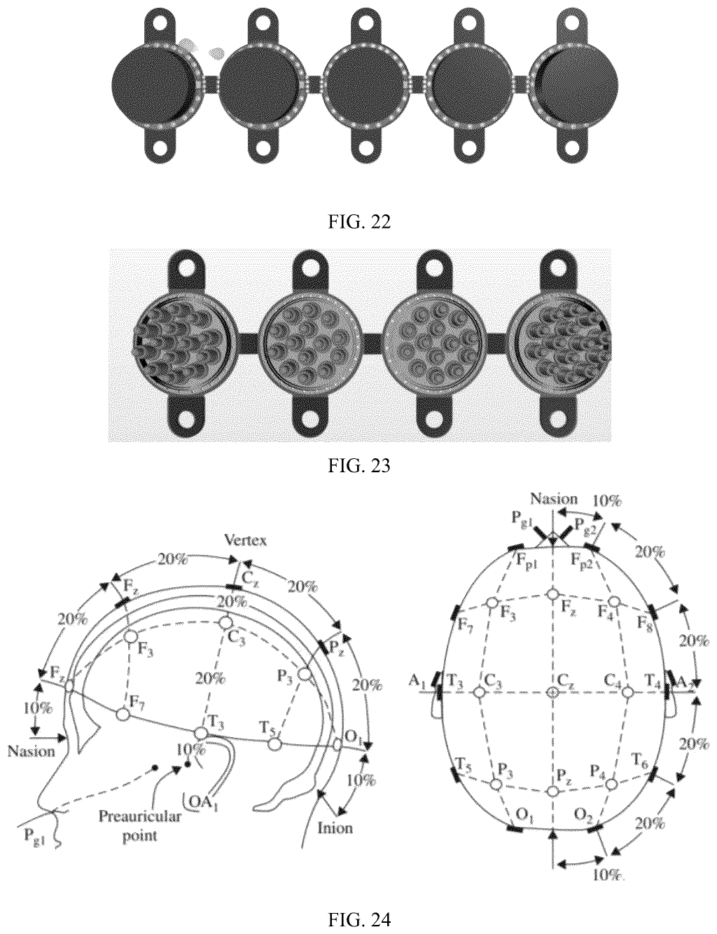

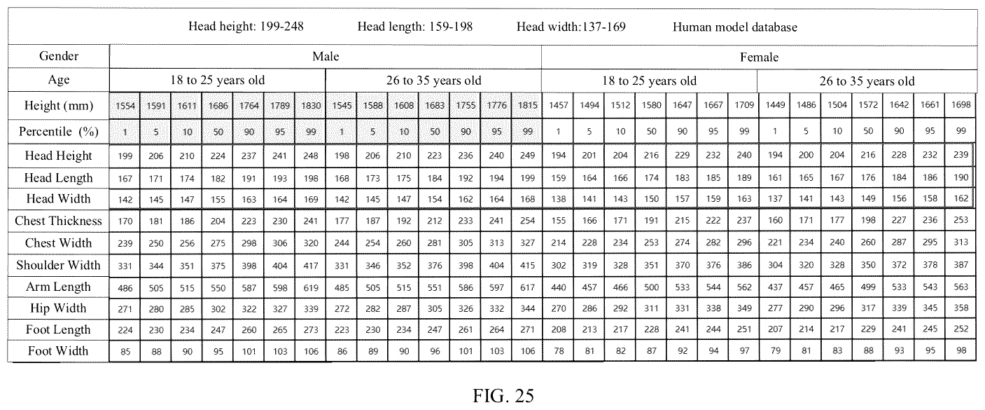

For example, the body in an embodiment illustrated in FIG. 18 adopts a soft, integrated elastic cap structure. A large circular sheet in the frontal lobe brain region represents the sheet-shaped dry electrode while a small circular sheet in the non-frontal lobe brain region represents the needle-shaped dry electrode (an electrode needle is located at a side in contact with a model scalp and is not illustrated). In the embodiments illustrated in FIG. 19 to FIG. 21, the main body adopts the integrated rigid structure.

An electrode cap (main body) of an electroencephalography apparatus may be made of a plurality of soft materials or forms to improve wearing comfort and a signal acquisition effect, such as a silicone material, cloth, an elastic material, etc. Silicone, featuring softness and elasticity, can fit a contour of the head and provide good wearing comfort. Also, the silicone material also possesses the characteristics of durability and easy cleaning. Soft fabrics or foam materials can be used to make the electrode cap, to provide a soft fit and breathability while reducing discomfort for a wearer. The electrode cap made of a material with certain elasticity can provide a certain degree of elasticity while maintaining stability, and adapt to people with different head circumferences. Regardless of the type of the soft material or the form selected, a key is to ensure that the electrode cap fits well with the head, maintains stable contact, and provides a comfortable wearing experience. A form of the electrode cap of the electroencephalography apparatus includes but is not limited to a mesh cap, a closed helmet, or a linear cap.

The electrode cap (main body) of the electroencephalography apparatus may also be made of a rigid material. The rigid material can better fix and support an electrode group, reduce the possibility of electrode position movement due to head movement, etc. Thus, stability of the signal acquisition is improved. The electrode cap made of the rigid material can better maintain consistency of shape and size, improving repeatability of the signal acquisition, and facilitating comparison and analysis of experimental data. The rigid material may be adjusted by means of stretching and contraction or pressurization to adapt to wearing requirements of different head shapes and improve the wearing comfort. Compared with the soft material, a surface of the rigid material is easier to clean, reducing the possibility of bacterial growth, and improving hygiene of wearing.

As illustrated in FIG. 18, and FIG. 19 to FIG. 21, in some embodiments, a non-frontal lobe electrode group includes a parietal lobe electrode group 4-3b and an occipital lobe electrode group 4-2b (the temporal lobe brain region may be distributed in any electrode group or a plurality of electrode groups). The parietal lobe electrode group 4-3b and the occipital lobe electrode group 4-2b both include the plurality of needle-shaped dry electrodes 42b. The parietal lobe electrode group 4-3b is mounted at a region of the main body corresponding to parietal lobe brain region of the user. The occipital lobe electrode group 4-2b is mounted at a region of the main body corresponding to the occipital lobe brain region of the user.

In addition to the acquisition electrode disposed at the frontal lobe brain region, the electroencephalography apparatus provided in the embodiments of the present disclosure also has acquisition electrodes disposed at the parietal lobe brain region, the occipital lobe brain region, and the temporal lobe brain region. In this way, brain signals from three main areas of the brain, namely a front area, a middle area, and a back area, can be obtained, achieving all-round brain signal acquisition. The brain signals from different regions exhibit distinct frequency and amplitude characteristics. Multi-region acquisition enhances spatiotemporal resolution of the brain signal, which facilitates more accurate reflection of changes in brain functional activity and also supports in-depth exploration of brain functions and associated mechanisms.

The multi-region acquisition also enables comparative analysis, such as comparisons among the frontal lobe brain region, the parietal lobe brain region, the occipital lobe brain region, and the temporal lobe brain region, which facilitates research on mutual influence and coordination among different brain regions. The brain signals from different regions are distinctive, and the multi-region acquisition can reduce interfering signals such as those from other muscles and eye movements, improving purity and reliability of acquisition.

In an embodiment of the present disclosure, the electrodes of the brain signal acquisition apparatus may all be dry electrodes. Compared with a wet electrode and a water electrode, which require gel and saline as conductive media to acquire the brain signal, the dry electrode can acquire the brain signal without any conductive medium. Therefore, a material selection and a shape of the dry electrode need to be more stringent. Due to an access of the conductive medium to the wet electrode and the water electrode, an electroencephalography contact impedance is reduced, noise of an input analog signal is reduced, and the brain signal can be better acquired. As for the dry electrode, a calculation formula of the contact resistance is as follows:

$${R}_{j} = \frac{K}{{( 0.102 F )}^{m}} ′ ,$$

where: K represents a material-related coefficient; F represents a contact pressure; and m represents a contact form, in which m=0.5 for point contact, m=0.5 to 0.7 for line contact, and m=1 for surface contact.

In an embodiment of the present disclosure, the electroencephalography apparatus may use an active electrode or a passive electrode. The active electrode has an amplifier integrated inside the active electrode to amplify the brain signal and suppress noise interference, improving a signal-to-noise ratio.

The passive electrode is usually more cost-effective than the active electrode, because the passive electrode does not require additional circuits and power supplies. The passive electrode is simple in structure and relatively easy to use.

In some embodiments, the sheet-shaped dry electrode 41b is a passive dry electrode. The sheet-shaped dry electrode 41b is constructed as a circular sheet with a predetermined thickness. A base material of the sheet-shaped dry electrode 41b is copper and a plating layer of the sheet-shaped dry electrode 41b is silver, or the base material of the sheet-shaped dry electrode 41b is copper and the plating layer of the sheet-shaped dry electrode 41b is silver-plated silver chloride. The electrode features a circular sheet design, which facilitates fixation and contact on a skin surface, achieving stable signal acquisition.

The electrode has the predetermined thickness, which helps ensure contact quality and stability between the electrode and the skin. Silver is an excellent conductive material, which is beneficial for guaranteeing good signal transmission and contact quality. In some cases, to further enhance electrical conductivity and corrosion resistance, a silver-plated silver chloride plating structure may be adopted.

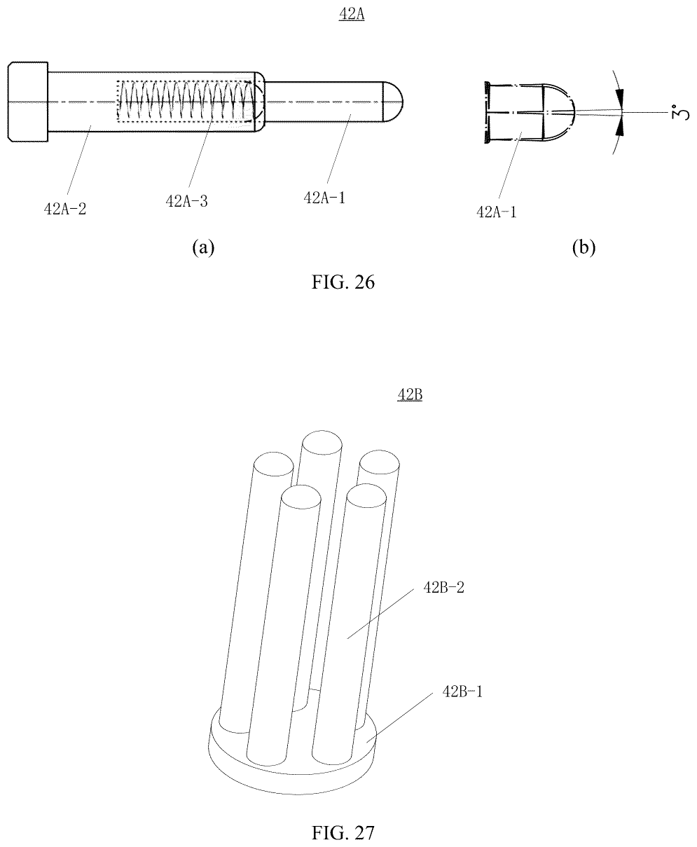

In some embodiments, as illustrated in FIG. 26 and FIG. 27, the needle-shaped dry electrode 42b is a passive dry electrode, and a structure of the needle-shaped dry electrode 42b may adopt various forms to achieve stable bioelectric signal acquisition and reduce interference to a tested object as much as possible.

For example, in an embodiment illustrated in FIG. 26, the needle-shaped dry electrode 42b is a pogo pin assembly 42A for a single acquisition point. The pogo pin assembly 42A includes an ejector pin 42A-1, a sleeve 42A-2, and a spring 42A-3 disposed in the sleeve 42A-2. The ejector pin 42A-1 is disposed in the sleeve 42A-2 via the spring 42A-3. The ejector pin 42A-1 has a working stroke that allows the ejector pin 42A-1 to extend and retract along an axis of the sleeve 42A-2. This structural design helps adapt to shapes of different skin surfaces and head circumference sizes of the human body, achieving better contact quality. The needle-shaped dry electrode 42b uses the copper as a base material and the silver as a plating layer. As the base material, the copper has excellent mechanical properties and electrical conductivity, while the silver of the plating layer helps ensure good signal transmission quality.

Further, relative to the fixed sleeve 42A-2, the ejector pin 42A-1 may also be arranged with a certain tilt or swing angle, such as 1° to 5°, or optionally 3°. The ejector pin 42A-1 with the certain tilt or swing angle enables the needle-shaped dry electrode 42b to better adapt to curve and shape of the skin surface, improving stability and quality of contact. This design exerts a positive influence on the signal acquisition effect in practical applications, especially in cases requiring long-term wearing, and can reduce electrode displacement or poor contact caused by movement or other factors. Such a swing design helps improve the stability and accuracy of the signal acquisition, meeting accuracy requirements for signal acquisition in fields such as biomedicine.

Further, the needle-shaped dry electrode 42b is the pogo pin assembly 42A for the single acquisition point, a maximum contact impedance of which at a working height is 50 mΩ, an elastic force at a normal working height ranges from 10 g to 30 g, a working stroke is 3.5 mm±0.02, and the working height is 6.5 mm±0.02.

For another example, in an embodiment illustrated in FIG. 27, the needle-shaped dry electrode 42b is the passive dry electrode. The needle-shaped dry electrode 42b is a claw-shaped electrode 42BB with a plurality of acquisition points. The claw-shaped electrode 42BB includes a base 42B-1 and a plurality of electrode needles 42B-2 disposed at the base 42B-1. The electrode needles 42B-2 are evenly distributed at a side of the base 42B-1. This design integrates the plurality of electrode needles 42B-2 at one single base 42B-1, facilitating arrangement of the electrode, enabling multi-point acquisition, and improving spatial resolution of the signal acquisition.

A design of the active dry electrode can effectively reduce noise interference caused by factors such as the electrode itself and a connection wire, while also providing signal amplification and regulation functions, all of which help improve quality of bioelectrical signal acquisition. The active electrode has a low input impedance, which can reduce the contact impedance between the electrode and the skin, and improve sensitivity of the signal acquisition. Due to a design of an internal amplifier, the active electrode has high immunity to interference from external power sources and the environment.

In some embodiments, the sheet-shaped dry electrode 41b and/or the needle-shaped dry electrode 42b are the active dry electrodes, and are disposed at a circuit board 43 including a voltage follower circuit. The voltage follower circuit at the circuit board 43 can help amplify and process a signal during the signal acquisition, improving the signal-to-noise ratio and ensuring signal accuracy. The voltage follower circuit can also be used to ensure signal stability under different conditions. For example, when a skin resistance or environmental interference changes, an operating parameter can be automatically adjusted to ensure the stability of the signal acquisition.

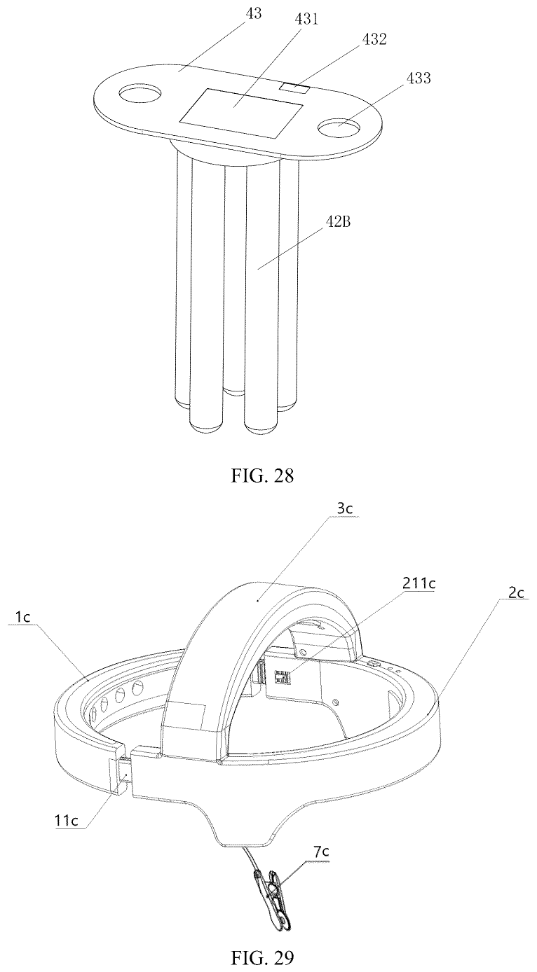

In an embodiment illustrated in FIG. 28, when the needle-shaped dry electrode 42b is an active dry electrode, the needle-shaped dry electrode 42b is a claw-shaped electrode 42B with the plurality of acquisition points. The claw-shaped electrode 42B includes the base 42B-1 and the plurality of electrode needles 42B-2 disposed at the base 42B-1. The electrode needles 42B-2 are evenly distributed at the side of the base 42B-1, and the base 42B-1 is welded to the circuit board 43. This design enables the plurality of acquisition points, improving the spatial resolution and the accuracy of the signal acquisition. Each electrode needle 42B-2 can be considered an independent acquisition channel, allowing simultaneous acquisition of bioelectrical activity from a plurality of signal sources. Welded attachment of the base 42B-1 ensures stability and reliability of the electrode, reducing signal distortion or interference caused by the electrode displacement or the poor contact.

Further, a shape of the circuit board 43 is designed to accommodate at least one claw-shaped electrode 42B. The circuit board 43 has at least one fixing hole (optionally two symmetrically arranged fixing holes for secure fixation) at the circuit board 43. In addition to a voltage follower circuit 431, the circuit board 43 may also have a solder pad 432 or a plug-in interface for connecting a signal line.

An output voltage of a voltage follower is the same as an input voltage of the voltage follower, that is, a voltage amplification factor of the voltage follower is constantly less than and close to 1. A significant feature of the voltage follower is that an input impedance is high and an output impedance is low. Generally speaking, it is easy to achieve the input impedance of several megohms. The output impedance is low, usually a few ohms, or even lower. A reason for using the voltage follower to design an active electroencephalography dry electrode is that an impedance of some materials of the dry electrode, when in contact with the scalp, is very high compared to the water electrode and the wet electrode. When using the dry electrode, an excessively high inherent impedance will cause the electrode to be in a Lead off state. Using the voltage follower circuit can reduce this impedance.

In some embodiments, one of the plurality of sheet-shaped dry electrodes 41b included in the frontal lobe electrode group 4-1b is a GND electrode. One of the sheet-shaped dry electrodes 41b is designated as the GND (ground) electrode, such as a front-most middle electrode. The GND electrode serves to provide a reference potential or serves as a point in a circuit for comparison and measurement of signals acquired by other electrodes.

The GND electrode can share a same physical contact location with other sheet-shaped dry electrodes 41b, for example, at a specific point on the scalp. By designating one sheet-shaped dry electrode 41b as the GND electrode, the signals acquired by the other electrodes can be compared with this electrode as a reference, and a potential difference relative to the ground can be calculated. Thus, more accurate measurement results can be obtained. In applications such as electroencephalography (EEG), a correct setup of the GND electrode is crucial for obtaining an accurate signal. Such setup helps reduce common-mode noise among the electrodes and provides a stable reference point, enabling the measurement results to be more reliable and comparable.

In an embodiment of the present disclosure, the frontal lobe electrode group 4-1b may be provided with five sheet-shaped dry electrodes 41b, of which four sheet-shaped dry electrodes 41b serve as electroencephalography activity electrodes, and one sheet-shaped dry electrode 41b serves as an electroencephalography GND electrode. Both the parietal lobe electrode group 4-3b and the occipital lobe electrode group 4-2b may be provided with four needle-shaped dry electrodes 42b. Of course, the needle-shaped dry electrode 42b described herein may adopt a structure of the claw-shaped electrode 42B described above, i.e., a single needle-shaped dry electrode 42b may also include the plurality of electrode needles 42B-2.

In an embodiment of the present disclosure, the number of electrodes is adjusted based on the brain regions that need to be acquired, and is not limited to a fixed number. Electrode positions can be arranged symmetrically in accordance with the international 10-20 system electrode placement method as illustrated in FIG. 24.

In some embodiments, the main body has a rigid structure. To increase applicability to different populations, the main body may be designed as a retractable structure. The main body includes a first semi-ring 1b corresponding to the parietal lobe brain region, the occipital lobe brain region, and the temporal lobe brain region, a second semi-ring 2b corresponding to the occipital lobe brain region, and a third semi-ring 3b corresponding to the parietal lobe brain region and/or temporal lobe brain region. The three semi-rings of the main body are interconnected to form an integrated structure. At least two of the first semi-ring 1b, the second semi-ring 2b, and the third semi-ring 3b have a retractable structure.

In another exemplary embodiment of the present disclosure, the non-frontal lobe electrode group is disposed at an inner side of the second semi-ring 2b and/or the third semi-ring 3b. Further, the frontal lobe electrode group 4-1b is disposed at an inner side of the first semi-ring 1b, the occipital lobe electrode group 4-2b is disposed at the inner side of the second semi-ring 2b, and the parietal lobe electrode group 4-3b is disposed at the inner side of the third semi-ring 3b. The first semi-ring 1b and the second semi-ring 2b form a closed ring. The third semi-ring 3b intersects with the first semi-ring 1b or the second semi-ring 2b. Relative to the second semi-ring 2b, the first semi-ring 1b and the third semi-ring 3b are both constructed as retractable structures.

In the above embodiments, the main body has the rigid structure configured to fix the electrode and fit the electrode tightly to the scalp for signal acquisition. In a design of the main body, the first semi-ring 1b and the third semi-ring 3b intersect with the second semi-ring 2b, and both the first semi-ring 1b and the third semi-ring 3b are designed as the retractable structures. The retractable structure may adopt a plug-in and locking structure with a certain depth. The plug-in and locking structure is typically designed as interlocking protruding and recessed shapes. When the two parts are inserted to a certain depth, a locking apparatus automatically secures the two parts together, ensuring that the main body will not loosen or shift due to external forces. This design is not only simple and easy to use, but also provides stable support and a comfortable experience during wearing. The main body is allowed to adapt to people with different head sizes and shapes, improving applicability and comfort of the main body. In addition, the retractable structure of the first semi-ring 1b and the retractable structure of the third semi-ring 3b also help alleviate a sense of pressure on the head when worn.

In another exemplary embodiment of the present disclosure, a dimension design of the first semi-ring 1b, the second semi-ring 2b, and the third semi-ring 3b of the above main body may refer to FIG. 8, which is a human model database. A dimension design of the main body may cover head circumference data of different genders, different ages, etc.

In some embodiments of present disclosure, the electroencephalography measurement apparatus is further provided. The apparatus has an integrated design to avoid interference from cable movement, enabling an entire apparatus to be more convenient to wear. The brain signal is a type of bioelectric signal that reflects electrical activity of brain neurons. When neurons in the brain fire electrical impulses, weak electrical currents are generated. These currents propagate through the scalp and brain tissue, and can be detected by electroencephalography electrodes. The brain signal typically appears as a periodic fluctuating pattern, whose characteristics include frequency, amplitude, morphology, and so on. Based on differences in frequency, the brain signals can be divided into different frequency bands, such as δ wave (0.5 Hz to 4 Hz), θ wave (4 Hz to 8 Hz), α wave (8 Hz to 13 Hz), β wave (13 Hz to 30 Hz), and γ wave (above 30 Hz).

The brain signal can provide information about brain activity, such as sleep state, level of consciousness, attention, cognitive processes, and emotional states. The brain signal has broad application value in clinical medicine, neuroscience research, and brain-computer interfaces. By analyzing the brain signal, brain functions and abnormalities can be revealed, helping to diagnose certain diseases or monitor brain health. The brain signal can also be used to study basic mechanisms of learning, memory, consciousness, and other cognitive processes.

Division of the brain regions is an important concept in neuroscience and neuroimaging, with different brain regions being responsible for different functions. Common brain regions include the prefrontal lobe brain region, the postfrontal lobe brain region, the parietal lobe brain region, the occipital lobe brain region and/or the temporal lobe brain region. The prefrontal lobe brain region is located in the front of the brain and is primarily involved in functions such as decision-making, behavior control, emotional regulation, and motor control. The postfrontal lobe brain region is located in the front of a top of the brain and is involved in functions such as spatial cognition, sensory information processing, attention, and spatial orientation. The parietal lobe brain region is located in the back of the top of the brain and is primarily responsible for processing and interpreting visual information. The occipital lobe brain region is located in the back of the brain and is involved in auditory processing, memory function, emotion recognition, and language comprehension. The temporal lobe brain region is located in a side of the brain and is responsible for some processing of visual information and face recognition.

In a first aspect, in some embodiments of the present disclosure, the electroencephalography measurement apparatus is provided (hereinafter referred to as an acquisition apparatus). In some embodiments, the acquisition apparatus includes at least two electrode group modules corresponding to different brain regions. Each of the at least two electrode group modules has a rigid semi-ring structure, the at least two electrode group modules being connected with each other to form an integrated structure. At least one of the at least two electrode group modules has a retractable structure, enabling the module to move in a direction towards or away from a center of the module to adjust a dimension of the module.

In the acquisition apparatus provided in the embodiments of the present disclosure, at least one electrode group module of the at least two electrode group modules has the retractable structure, which can adjust a fitting degree between the electrode group module and a corresponding brain region, to adapt to the subjects with different head circumferences. This method can also adjust a contact pressure between the electrode and the brain skin. An appropriate contact pressure can improve the contact quality between the electrode and the skin, thereby reducing the contact resistance.

The contact pressure refers to a pressure exerted by the electrode on the skin surface. An excessively low contact pressure may cause a gap between the electrode and the skin, increasing the contact resistance; while an excessively high contact pressure may damage the skin or cause discomfort.

Currently, most common brain signal acquisition apparatus available on the market adopt a soft cap structure. During wearing, the electrode is not only difficult to align properly but also prone to damage from pulling or interference with the brain signal acquisition. The soft cap structure does present certain challenges in wearing. Due to differences in head shapes of different subjects, the wearer may need to adjust a position and a tightness of a cap to ensure the electrode fit the scalp. This adjustment process is not entirely accurate, which may result in incorrect electrode alignment and inconsistent electrode contact pressure among different subjects, ultimately affecting the accuracy and the quality of the signal acquisition. Additionally, pulling, friction, and other issues may occur during wearing, which may disrupt the contact between the electrode and the scalp, resulting in signal distortion or loss. Further, wearing a soft cap for a long time may also cause discomfort and a sense of pressure, affecting the comfort of the user.

Compared with the soft cap structure, three module components in the embodiments of the present disclosure may all adopt a rigid structure, that is, the brain signal acquisition apparatus in the embodiments of the present disclosure adopts an adjustable rigid cap, which can better solve a problem of inaccurate electrode alignment. Since the adjustable hard cap can be adjusted in size based on the shape of the head of the user, an accurate position and correspondence of the electrodes can be better guaranteed. In this way, the accuracy and precision of brain signal acquisition can be improved, better reflecting a state of the brain activity. The adjustable rigid cap adopts a retractable adjustment structure, which enables the apparatus to adapt to people with different head circumferences. The user can adjust the apparatus based on his own head circumference to obtain a more comfortable and suitable wearing experience.

Secondly, the adjustable rigid cap has better stability and durability. A rigid cap can provide better support and protection, better protecting the electrode and a sensor from external physical interference. Also, the rigid cap can prevent deformation or damage during wearing, extending a service life and performance of a device. In addition, a rigid housing structure can provide better support and stability, allowing the electrode to be more easily and accurately aligned with a specific position on the head during wearing. In this way, good contact between the electrode and the scalp is ensured, enhancing the precision and the accuracy of the signal acquisition.

Finally, the rigid cap can improve the comfort of the wearer. Since the rigid cap can be customized or adjusted based on a head shape of the wearer, which can better fit the head shape, and reduce the sense of pressure and the discomfort. In this way, user experience of the wearers can be improved, facilitating their acceptance and use of the brain signal acquisition apparatus, and facilitating promotion of the brain signal acquisition apparatus. In addition, the rigid housing structure is generally more portable and easier to use than the soft cap structure. Thus, the user can place the apparatus in a carry-on bag or a case, facilitating the brain signal acquisition in different occasions.

The brain signal acquisition apparatus in the embodiments of the present disclosure adopts the adjustable rigid cap structure, which has better electrode alignment accuracy, stability, durability and comfort, and can be used to improve performance and the user experience of the brain signal acquisition apparatus.

In some embodiments, each electrode group module may be provided with the same or different electrodes, and the plurality of electrodes in an electrode group may also adopt the same or different electrodes. At least one electrode group module of the electrode group modules may be provided with a plurality of brain signal acquisition electrode groups of the same type; alternatively, at least one electrode group module of the electrode group modules may be provided with at least two different types of brain signal acquisition electrode groups.

It should be understood that the type mentioned here may refer to a form, such as the form of electrode needle or electrode sheet, or the dry electrode and the wet electrode, or those used for acquiring different types of brain signals. The apparatus can be used for multiple purposes and applications. For example, in some studies, to obtain more extensive information on electroencephalographic activity, the plurality of electrode groups of the same type may be used to expand coverage of data acquisition. If an electrode group module is provided with at least two different types of brain signal acquisition electrode groups, neural activity data at different levels and resolutions can be simultaneously recorded, providing a more comprehensive understanding and analysis of brain functions and activity.

In some embodiments, the brain signal acquisition electrode group includes an electroencephalography EEG acquisition electrode. The EEG acquisition electrode is used to record electrical activity in cerebral cortex. The acquisition electrode may be placed on the scalp to measure an electrical signal of the brain activity. The acquisition electrode can capture the electrical activity of neurons in the brain and convert the electrical activity into a digital signal that can be used for analysis and research. By analyzing an EEG signal, activity patterns of the brain in different states can be understood, such as sleep, attention, cognitive tasks, etc.

In the above embodiments, in addition to the electroencephalography (EEG) acquisition electrode, other types of electrodes may also be used for the brain signal acquisition, such as an electrocorticography (ECoG) acquisition electrode, a computed tomography (CT) electrode, and a deep brain stimulation (DBS) electrode. A CoG electrode is an electrode that is directly implanted on a surface of the brain or placed under dura mater. Compared to EEG, the ECoG can provide higher spatial resolution and signal quality, and can record more detailed neural activity. The CT electrode is an electrode used for positioning and navigation inside the brain tissue and is commonly used in neurosurgery. A computed tomography (CT) technology allows for accurate positioning, allowing brain signal acquisition in specific areas. The DBS electrode is an electrode implanted deep within the brain and is used to treat neurological disorders such as Parkinson's disease and depression. In addition to a stimulation function, the DBS electrode can also be used to record the brain signal to monitor treatment efficacy.

In some embodiments, the electroencephalography EEG acquisition electrode includes an EEG dry electrode. The EEG dry electrode includes a base material and a plating layer. The base material is made of copper, and the plating layer is made of silver. A dry electrode using a copper base material and a silver plating layer may provide excellent electrical conductivity and signal quality. The copper base material has excellent mechanical strength and electrical conductivity, allowing for stable attachment to the scalp. The silver plating layer has a low electrical resistance and better electrical conductivity, which can reduce signal loss and improve the signal quality.

In some embodiments, the brain signal acquisition electrode group includes a functional near-infrared spectroscopy FNIRS light source, an emitter, and a detector. Functional Near-Infrared Spectroscopy (FNIRS) is a non-invasive neuroimaging technique used to measure changes in cerebral blood oxygen levels. The functional near-infrared spectroscopy (FNIRS) light source uses a near-infrared light source to emit invisible light into scalp tissue. The light source can use infrared or near-infrared light (with a wavelength ranging from 650 nanometers to 1000 nanometers), which can penetrate the skull and most soft tissues. The emitter is an assembly that directs light from the light source to a measured area. The emitter may include one or more fiber optical bundles that direct light emitted by the light source to a specific brain region. The detector is configured to measure light reflected or transmitted back from the measured area and may be composed of a photosensitive detector (such as a photodiode) to measure an intensity of the reflected or transmitted light.

In some embodiments, the brain signal acquisition electrode group includes an anode electrode and a cathode electrode used for transcranial direct current stimulation (TDCS). Transcranial Direct Current Stimulation (TDCS) is a technique that modulates the brain activity by delivering a weak direct current through the anode electrode and the cathode electrode placed on the scalp. In the TDCS, the anode electrode and the cathode electrode may be placed on the scalp to apply the current to specific brain regions. The anode electrode, typically a positive electrode, enhances the current in an underlying brain region, thereby promoting neuronal excitation. The cathode electrode, typically a negative electrode, inhibits the current in the underlying brain region, leading to neuronal inhibition.

In some embodiments, the electrode group modules include at least two of a first electrode group module corresponding to the prefrontal lobe brain region and/or the postfrontal lobe brain region, a second electrode group module corresponding to the occipital lobe brain region, and a third electrode group module corresponding to the parietal lobe brain region and/or the temporal lobe brain region. The frontal lobe brain region, the postfrontal lobe brain region, the occipital lobe brain region, the parietal lobe brain region, and the temporal lobe brain region are common target brain regions, and different combinations of electrode groups can be used to acquire signals from the target brain regions. Selection of electrode combinations and specific placement positions vary based on the research or treatment purposes. For example, to explore an effect of the prefrontal lobe brain region on a cognitive function, the first electrode group module placed in the prefrontal lobe brain region and/or the postfrontal lobe brain region may be used; and to explore an effect of the parietal lobe brain region on a motor function, the third electrode group module placed in a top and a center area may be used. A specific electrode combination and a placement position need to be determined based on a specific experimental design, research hypothesis, and treatment needs.

In an embodiment of the present disclosure, the brain signal acquisition apparatus may adopt a combination of any two electrode group modules, or a combination of three electrode group modules. Taking the three electrode group modules as an example, a specific structure is explained in detail.

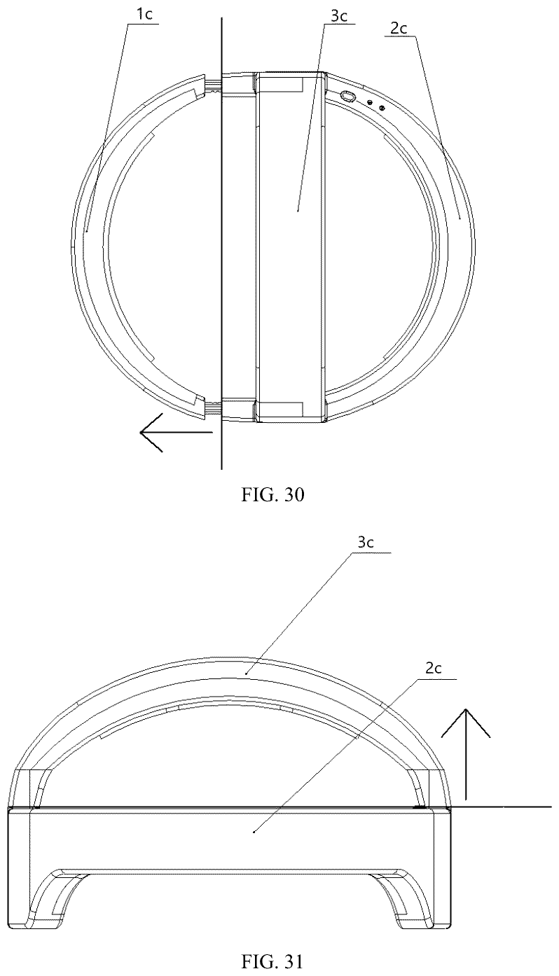

In some embodiments, as illustrated in FIG. 29 to FIG. 30, the electroencephalography measurement apparatus includes a first electrode group module 1c, a second electrode group module 2c, and a third electrode group module 3c. An overall structure of these three modules is significantly different from a cap body in the existing art. A cap body of an electroencephalography device in the existing art is mostly composed of a mesh-shaped flexible material, while a housing of the first electrode group module 1c, a housing of the second electrode group module 2c, and a housing of the third electrode group module 3c in the present disclosure are all made of rigid materials, which enables a wearing process to be simple and convenient, and greatly reduces required time.

Further, the first electrode group module 1c, the second electrode group module 2c, and the third electrode group module 3c all have a rigid semi-ring structure, and the three modules are interconnected to form an integrated structure. The first electrode group module 1c and the second electrode group module 2c form a closed ring. The third electrode group module 3c is configured to intersect with the first electrode group module 1c or the second electrode group module 2c. In another exemplary embodiment of the present disclosure, the closed ring formed by the first electrode group module 1c and the second electrode group module 2c is located in a same plane, in such a manner that a front and a back are symmetrical, an appearance is more aesthetically pleasing, and the arrangement of the electrode is more convenient. However, the closed ring is not limited thereto. The first electrode group module 1c and the second electrode group module 2c may also be slightly angled to adapt to different application scenarios.

Further, at least one of the first electrode group module 1c, the second electrode group module 2c, and the third electrode group module 3c has a retractable structure, enabling the module to move in a direction towards or away from a center of the module to adjust a dimension of the module.

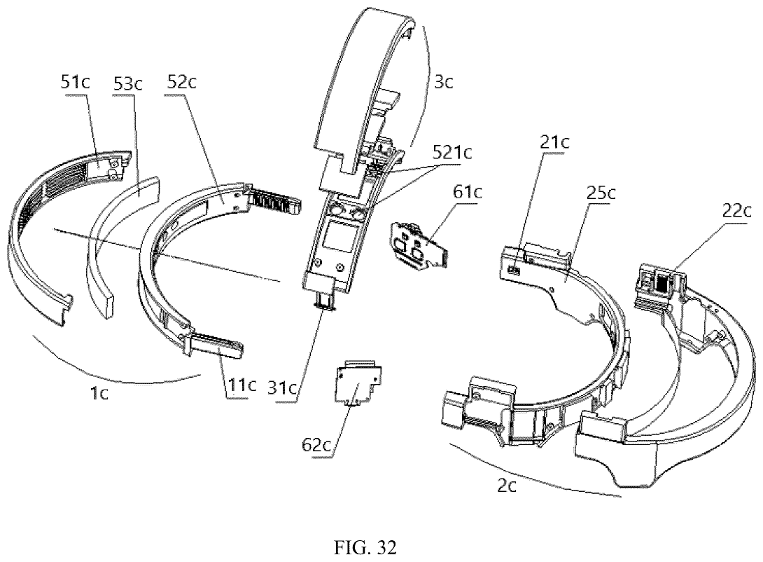

In some embodiments, as illustrated in FIG. 32 to FIG. 34, a first plug-in structure is formed at a connection part of the first electrode group module 1c and the second electrode group module 2c. At least one end of one module of the first electrode group module 1c and the second electrode group module 2c is formed with a protruding first plug-in block 11c, and a corresponding end of the other module of the first electrode group module 1c and the second electrode group module 2c has a recessed first plug-in groove 21c, the first plug-in block 11c being configured to move in the first plug-in groove 21c to achieve extension and retraction.

A design of the first plug-in blocks 11c at both ends of the “first electrode group module 1c” is further explained as an example. An end of the first electrode group module 1c has a protruding plug-in block, and a corresponding end of the second electrode group module 2c has a recessed plug-in groove. A purpose of this design is to achieve an extension and retraction function between modules. When the plug-in block is inserted into the plug-in groove, the plug-in block is configured to move in the plug-in groove, in such a manner that an entire connection part can extend and retract. This extension and retraction design can adjust a distance or an angle between two modules as needed, to adapt to different usage requirements or head sizes. Such a structural design can provide better adaptability and comfort, allowing the electroencephalography measurement apparatus to better fit the shape of the head and provide more reliable signal contact. Also, during use, the user can freely adjust a length or an angle of the connection part as needed to obtain a better user experience and signal quality.

In some embodiments, the first plug-in structure further includes a first tightening structure for locking the first plug-in block 11c in the first plug-in groove 21c. The first tightening structure includes: a plurality of first limit grooves 111c arranged in a first direction and formed at the first plug-in block 11c, each of the plurality of first limit grooves 111c extending in a second direction, the first direction intersecting with the second direction, and the first direction being an extension and retraction direction of the first electrode group module 1c or the second electrode group module 2c; and a first locking tongue member 211c formed at a side of the first plug-in groove 21c and fixedly arranged, a body of the first locking tongue member 211c being elastic, and the first locking tongue member 211c having a first protruding end 2111c. When the first protruding end 2111c is clamped in one of the plurality of first limit grooves 111c, the first electrode group module 1c and the second electrode group module 2c are clamped tightly to each other.

Each of the plurality of first limit grooves 111c extends in the second direction. The first direction intersects with the extension and retraction direction of the first electrode group module 1c or the second electrode group module 2c. These limit grooves function to provide positional constraints to ensure a movement range of the plug-in block in the plug-in groove. The body of the first locking tongue member 211c is elastic. The first locking tongue member 211c has the first protruding end 2111c. When the first protruding end 2111c is clamped in one of the plurality of first limit grooves 111c, the first electrode group module 1c and the second electrode group module 2c are clamped tightly to each other. In other words, the first protruding end 2111c can be embedded in a limit groove and locked therewith, ensuring a stable connection. Through the above design, the first plug-in block 11c is extensible and retractable in the first plug-in groove 21c, and is locked to a specific position of the plug-in groove by the first tightening structure. This locking mechanism can maintain stability of the plug-in block and prevent the plug-in block from loosening or falling off during use.

In some embodiments, a second plug-in structure is formed at a connection part of the third electrode group module 3c and the first electrode group module 1c or the second electrode group module 2c. At least one end of one module is formed with a protruding second plug-in block 31c, and a corresponding end of the other module has a recessed second plug-in groove 22c, the second plug-in block 31c being configured to move in the second plug-in groove 22c to achieve extension and retraction.

A further detailed explanation will be given by taking the third electrode group module 3c provided with the second plug-in block 31c as an example. The protruding second plug-in blocks 31c are formed at both ends of the third electrode group module 3c, while the corresponding end of the second electrode group module 2c has the recessed second plug-in groove 22c. With this design, the second plug-in block 31c is configured to move in the second plug-in groove 22c, enabling the extension and the retraction between the modules. The second plug-in block 31c has a protruding shape, and is configured to be inserted into or pulled out of the second plug-in groove 22c. The recessed second plug-in groove 22c is configured to accommodate the second plug-in block 31c and provide functions of fixing and positioning. By adjusting a position of the second plug-in block 31c in the second plug-in groove 22c, the extension and the retraction between the modules can be achieved to adapt to different usage requirements.

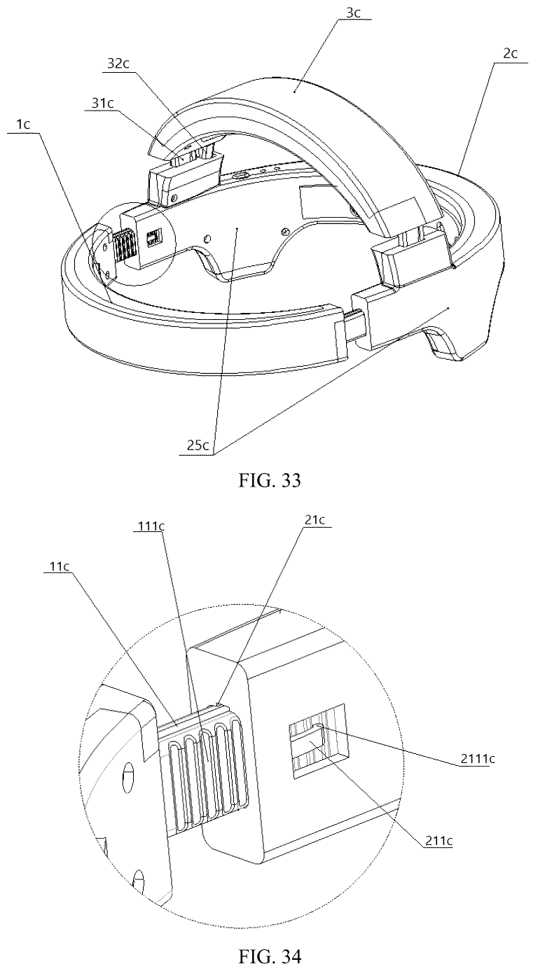

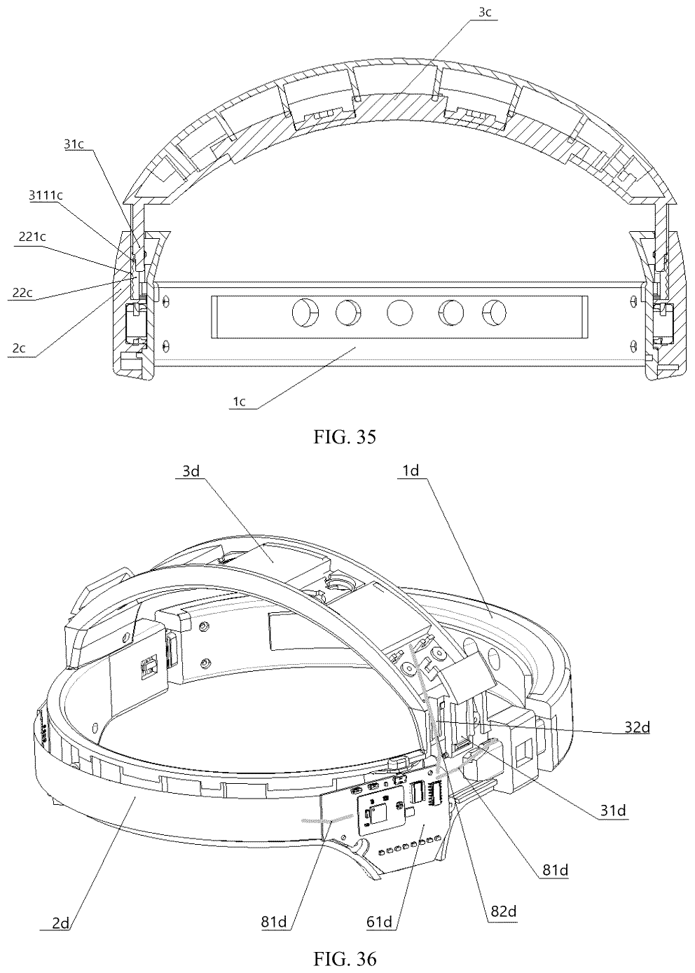

In some embodiments, as illustrated in FIG. 33 to FIG. 29, the second plug-in structure further includes a second tightening structure for locking the second plug-in block 31c in the second plug-in groove 22c. The second tightening structure includes: a plurality of second limit grooves 221c arranged in a third direction and formed at the second plug-in groove 22c, each of the plurality of second limit grooves 221c extending in the first direction, the third direction intersecting with the first direction, and the third direction being an extension and retraction direction of the third electrode group module 3c; and a second locking tongue member 311c formed at a side of the second plug-in block 31c and fixedly arranged, a body of the second locking tongue member 311c being elastic, and the second locking tongue member 311c having a second protruding end 3111c. When the second protruding end 3111c is clamped in one of the plurality of second limit grooves 221c, the third electrode group module 3c is clamped tightly with the first electrode group module 1c or the second electrode group module 2c.

The plurality of second limit grooves 221c are arranged in the third direction and formed at the second plug-in groove 22c. Each of the plurality of second limit grooves 221c extends in the first direction. The third direction intersects with the extension and retraction direction of the third electrode group module 3c. These second limit grooves 221c function to provide positional constraints to ensure a movement range of the second plug-in block 31c in the second plug-in groove 22c. The body of the second locking tongue member 311c is elastic. The second locking tongue member 311c has the second protruding end 3111c. When the second protruding end 3111c is clamped in one of the plurality of second limit grooves 221c, the third electrode group module 3c is clamped tightly with the first electrode group module 1c or the second electrode group module 2c. In other words, the second protruding end 3111c can be embedded in a limit groove and locked therewith, ensuring a stable connection. Through the above design, the second plug-in block 31c is extensible and retractable in the second plug-in groove 22c, and is locked to a specific position of the plug-in groove by the second tightening structure. This locking mechanism can maintain stability of the plug-in block and prevent the plug-in block from loosening or falling off during use.

In some embodiments, as illustrated in FIG. 32, an end of the first plug-in block 11c extends forward, forming a self-guided structure. A co-directing guide post 32c is disposed near the second plug-in block 31c at an end of the third electrode group module 3c. These structural designs can ensure a smoother extension and retraction process. In addition, limit mechanisms may be disposed at both ends of a stroke between the first plug-in block 11c and the first plug-in groove 21c, and between the second plug-in block 31c and the second plug-in groove 22c to prevent a structural member from falling off or being damaged.

In some embodiments, the first electrode group module 1c, the second electrode group module 2c, and the third electrode group module 3c are each provided with a plurality of dry electrodes. Currently, common electroencephalography acquisition methods on the market are divided into three types: a gel electrode, a water electrode, and a dry electrode. The gel electrode and the water electrode both require scalp pretreatment before the brain signal acquisition. The dry electrode can avoid inconvenience of the scalp pretreatment and can be applied in various natural scenarios more quickly and flexibly.

As an implementable method, the second electrode group module 2c is provided with a three-way connection portion 25c at an intersection of the three modules, which can not only be engaged with the first electrode group module 1c, but also be engaged with the third electrode group module 3c, realizing functions of the first electrode group module 1c and the third electrode group module 3c. In addition, the three-way connection portion 25c may also be provided with a function board 61c and a second power board 62c. The function board 61c is responsible for the acquisition, the processing, and the analysis of the brain signal, while the second power board 62c provides power supply and corresponding power management functions. A synergistic effect of the function board 61c and the second power board 62c enables the electroencephalography measurement apparatus to operate effectively and provide accurate and reliable electroencephalography data.

In some embodiments, each module includes a second curved housing 51c, a second curved housing 51c, and a second intermediate layer 53c. The second curved housing 51c has a second electrode hole 521c for fixing the electrode. The second intermediate layer 53c is disposed between the second curved housing 51c and the second curved housing 51c, and the second curved housing 51c is located in an inner layer of the second curved housing 51c. The second intermediate layer 53c is elastic or is configured to mount wires that achieve electrical connection of the respective electrodes. Each electrode is mounted in the second electrode hole 521c of the second curved housing 51c through an elastic member. The first electrode group module 1c, the second electrode group module 2c, or the third electrode group module 3c is provided with a GND electrode. The elastic member may be a spring or an elastic pad.

In the electroencephalography measurement apparatus provided in the embodiments of the present disclosure, the first electrode group module 1c may be provided with four sheet-shaped active electrodes and one sheet-shaped GND electrode, the second electrode group module 2c may be provided with four needle-shaped active electrodes, and the third electrode group module 3c may be provided with four needle-shaped active electrodes, for a total of 12 channels of data. The number of electrodes in the electroencephalography measurement apparatus in the embodiments of the present disclosure can be adjusted arbitrarily, and the number of data channels can be correspondingly set to various numbers such as 16 channels, 32 channels, etc.

The GND electrode is an electrode connected to the ground, serving as a grounding point in a circuit. The GND electrode is typically used to establish a reference potential or provide a reference point for a loop, to ensure a normal operation of the circuit and stability of signal transmission. Components and wires connected to the GND electrode will have a same potential, ensuring sharing and balance among various parts of the circuit. The GND electrode can provide a reference potential for the circuit, allowing signals to be transmitted reliably and consistently. The GND electrode can safely release static electricity or electromagnetic interference in the circuit. The GND electrode can protect human safety by connecting a metal casing or contact parts of the circuit to the ground to avoid a risk of electric shock. The GND electrode can provide protection against circuit noise and interference, directing the noise and the interference to the ground without interfering with other parts. The GND electrode serves as a loop path for the circuit, ensuring a current to flow normally in the circuit.

As illustrated in FIG. 29, the electroencephalography measurement apparatus further includes a second ear clip 7c serving as a reference electrode. The second ear clip 7c is connected to the main body of the electroencephalography measurement apparatus through an ear clip wire. An ear clip of the electroencephalography measurement apparatus is used as the reference electrode to provide a reference point for correcting brain signal measurement. The brain signal is measured using the electrodes on the scalp, which are typically located at different positions on the head. In electroencephalography (EEG) measurement, a reference electrode is a technique used to eliminate interference from environmental and physiological noise on the brain signal. The reference electrode works by placing an electrode at a position unrelated to the brain signal, and the position is used as the reference point for calibration. By attaching a clip electrode to ears or behind the ears, a stable reference potential can be obtained, which is not easily affected by external noise. In this way, common noise in the brain signal can be reduced, and accuracy and reliability of measurement can be improved.

It should be understood that, the electroencephalography measurement apparatus in the embodiments of the present disclosure only illustrates the second ear clip 7c in FIG. 29. In other figures, the ear clip is omitted or the reference electrode is set in other ways, such as a rubber disc electrode or an elastic band electrode.

In some embodiments, the first electrode group module 1c includes a plurality of electrodes in the form of a second electrode sheet 41c. A certain relationship exists between an electrode dimension and the contact resistance. Generally speaking, when the electrode dimension is large, the contact resistance is relatively small; and when the electrode dimension is small, the contact resistance is relatively large. This is because an increase in the electrode dimension can increase a contact area between the electrode and the skin, in such a manner that a path length of the current is reduced, and a resistance encountered by the current at a contact interface is reduced. Conversely, a decrease in the electrode dimension will lead to a decrease in the contact area, an increase in the path length of the current, and an increase in the resistance encountered by the current at the contact interface.

In some embodiments, the plurality of electrodes included in the second electrode group module 2c and the third electrode group module 3c are in the form of a second electrode needle 43c.

Portable dry electrodes used in the electroencephalography measurement apparatus come in two forms: one is the second electrode sheet 41c, and the other is the second electrode needle 43c. The second electrode needle 43c may adopt a spring design to provide a constant and comfortable pressure, which enhances contact between the electrode and the skin while reducing motion artifacts. With the signal quality comparable to that of a wet-electrode electroencephalography system, the second electrode needle 43c is currently mainly used in neurofeedback, brain-computer interface, and psychological research. The electrode utilizes an active/passive shielding technology to prevent electromagnetic interference.

In conjunction with the existing art, a calculation formula of the contact resistance is:

$${R}_{j} = \frac{K}{{( 0.102 F )}^{m}} ,$$

where: Rj represents a contact resistance; K represents a material-related coefficient, such as tin-plated copper or silver-plated copper; F represents a contact pressure, with the unit of Newton(s); and m represents a contact form, in which m=0.5 for point contact, m=0.5 to 0.7 for line contact, and m=1 for surface contact.

Generally, a metal electrode (such as a silver/silver chloride electrode) has a low contact resistance, which can provide good signal quality and low electrode noise. Other materials such as stainless steel and carbon nanotubes can also be used for electrode production, but their contact resistance may be higher than that of the metal electrode.

In an embodiment of the present disclosure, the dry electrode includes a base material and a plating layer. The base material is made of copper, and the plating layer is made of silver. Selection of a contact electrode material has a significant impact on the contact resistance. The dry electrode in the present disclosure uses a silver-plated copper material, which has advantages such as cost, electrical conductivity, conductive stability, oxidation resistance, and biocompatibility. The copper, as a base material, has good electrical conductivity and can effectively transmit the brain signal. High electrical conductivity of the silver makes the silver an excellent plating material, further improving the electrical conductivity of the electrode. In this way, the resistance is reduced, allowing for better capture of weak brain signals. A silver-plated copper electrode has excellent electrical stability and can maintain a certain level of conductivity over a long period of time, which is crucial for long-term electroencephalography monitoring and experimental research, ensuring the stability and the reliability of the signal. The silver plating layer has as an antioxidant effect, which reduces effects of oxidation on an electrode material. Oxidation may cause corrosion on an electrode surface or form an insulation layer, which affects transmission and quality of the signal. The silver plating layer protects a copper electrode from oxidation damage, extending a service life of the copper electrode. The silver-plated copper electrode is biocompatible with the skin and the scalp, and does not cause severe allergic or irritating reactions, which is crucial for long-term wearing of the electrode, human experiments, and clinical applications.

It should be noted that, although the silver-plated copper electrode has these advantages, correct electrode placement, stable contact, and complete and detailed data processing and analysis are also key factors in ensuring the quality of the brain signal.

To solve or alleviate problems such as electrode cable assembly in a soft cap electroencephalography apparatus in the existing art, in some embodiments of the present disclosure, the electroencephalography measurement apparatus is further provided. The acquisition apparatus integrates electrode cables into a rigid main body, and there is no need to assemble or disassemble the electrode cables before and after use, which greatly facilitates the use of the electroencephalography apparatus, improves acceptance of the user, and provides convenience for large-scale promotion of the electroencephalography apparatus.

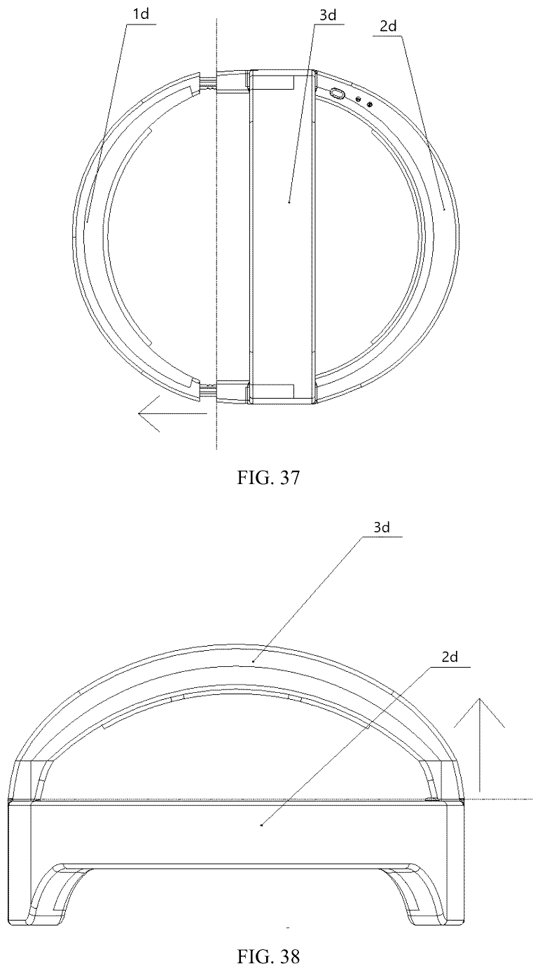

In some embodiments of the present disclosure, the electroencephalography measurement apparatus is further provided. As illustrated in FIG. 36 to FIG. 40, the acquisition apparatus includes a main body, a brain signal mainboard card 61d, a plurality of electrodes, and a signal transmission line that are integrally disposed at the main body.

In the existing art, the electroencephalography apparatus in the form of a soft cap needs to be able to fit the scalp stably and maintain good contact quality to ensure the accuracy and the stability of the signal acquisition. However, due to the differences in head shapes and sizes, fitness and stability of the soft cap may pose certain challenges. The soft cap needs to remain comfortable when worn for a long period of time to meet the needs of long-term brain signal acquisition. However, some electroencephalography apparatuses in the form of the soft cap may cause discomfort to the wearer due to improper material selection or design.

Compared with the soft cap structure, in an embodiment of the present disclosure, the main body has the integrated rigid structure having the wiring channel formed inside or at the surface of the main body. The rigid main body can provide better support and protection, better protecting the electrode and the sensor from the external physical interference. Also, the rigid main body can prevent deformation or damage during wearing, extending the service life and performance of the device. The rigid main body can improve the comfort of the wearer. Since the rigid main body can be customized or adjusted based on the head shape of the wearer, which can better fit the head shape, and reduce the sense of pressure and the discomfort. In this way, user experience of the wearers can be improved, facilitating their acceptance and use of the brain signal acquisition apparatus, and facilitating the promotion of the brain signal acquisition apparatus.

In an embodiment of the present disclosure, the plurality of electrodes are grouped and arranged in at least one brain signal acquisition area of the main body. The wiring channel of the main body extends from the brain signal acquisition area to an area of the main body for mounting the brain signal mainboard card 61d, and is configured to embed the signal transmission line. The signal transmission line is configured to connect the plurality of electrodes and the brain signal mainboard card 61d, to transmit a brain signal acquired by the plurality of electrodes.

In the above embodiments, the electroencephalography measurement apparatus integrates the brain signal mainboard card 61d, the plurality of electrodes, and the signal transmission line into an integrated rigid structure, providing a more convenient user experience. Arrangement of the wiring channel allows the signal transmission line to form a channel inside or at the surface of the main body, resulting in better neatness and easier management.

In addition, the electrodes are grouped and arranged in the brain signal acquisition area, which can effectively capture the brain signals in each area. Also, extension of the wiring channel allows the signal transmission line to be connected to the electrode and the brain signal mainboard card 61d, in such a manner that the acquired brain signal is transmitted. This design tightly integrates various parts of the brain signal acquisition apparatus, providing a more efficient and convenient solution for the brain signal acquisition.

In an embodiment of the present disclosure, the electroencephalography measurement apparatus can shorten experimental preparation time. All the transmission lines are disposed inside the main body of the apparatus, which can shield electromagnetic crosstalk in some environments. The entire apparatus adopts an integrated design, unlike a traditional electroencephalography apparatus which consists of separate cables and cap structures and which requires assembling the cables and the cap before each test. In this way, experimental preparation time is greatly shortened, and post-experiment equipment arrangement steps are eliminated.

In an embodiment of the present disclosure, the main body has the integrated rigid structure. A rigid structure can effectively protect internal electronic components while also maintaining an overall shape and stability of the main body, enabling the entire apparatus to be easier to carry and use. An integrated structure avoids a risk of cable disconnection caused by the transmission line being exposed to the outside. Due to an integrated design, the main body itself is a complete apparatus, which is convenient for the user to carry. The user can put the main body in a dedicated bag or case for easy portability without worrying about the loss or damage of parts. Due to stability of the rigid structure, the user can quickly put on and start a cap-type brain signal acquisition apparatus when needed, without a need for a complicated assembly process, improving convenience and efficiency of use.

In addition, the main body has a certain weight and pressure, which can help the electrode fit more closely to the brain area to be measured.

A design concept of the electroencephalography measurement apparatus in the embodiments of the present disclosure aligns with user needs and delivers better user experience and functionality.

In some embodiments, the brain signal acquisition area of the main body includes one or more of a prefrontal lobe brain signal acquisition area, a postfrontal lobe brain signal acquisition area, a parietal lobe brain signal acquisition area, an occipital lobe brain signal acquisition area, and a temporal lobe brain signal acquisition area.

Based on an anatomical structure and functional divisions of the human brain, the prefrontal lobe brain region is located at the front of forebrain and is associated with functions such as cognitive control, decision-making, and emotional regulation. The postfrontal lobe brain region is located at the back of the forebrain and is associated with functions such as motor control and sensory information processing. The parietal lobe brain region is located at the top of the brain and is associated with functions such as vision and spatial cognition. The occipital lobe brain region is located at the back of the brain and is associated with functions such as visual information processing. The temporal lobe brain region is located at the side of the brain and is associated with functions such as hearing, memory, and language.

In an embodiment of the present disclosure, the main body of the acquisition apparatus includes at least one signal acquisition area for the above brain regions. By placing corresponding electrodes in these brain signal acquisition areas, signals such as electrical activity, blood oxygenation levels, or magnetic fields from different brain regions can be acquired, in such a manner that brain functions and cognitive processes can be studied, which is of great significance for understanding working principles of the brain and developing applications such as brain-computer interfaces.

In some embodiments, at least one of the brain signal acquisition areas is provided with a plurality of brain signal acquisition electrodes of the same type; alternatively, at least one of the brain signal acquisition areas is provided with at least two different types of brain signal acquisition electrodes. In this embodiment, a plurality of brain signal acquisition electrodes of the same type may be disposed in the same brain signal acquisition area, or a plurality of different types of brain signal acquisition electrodes may be disposed in the same brain signal acquisition area. The type described herein may include electrodes of different shapes, different principles, and wet or dry electrodes.

In some embodiments, the electrodes disposed in at least one of the brain signal acquisition areas include an electroencephalography EEG acquisition electrode. The electroencephalography (EEG) acquisition electrode is used to record changes in electrical potential resulting from the electrical activity of the neurons in the cerebral cortex. EG electrodes are placed on the surface of the scalp to acquire signals from electroencephalographic activity. These electrodes are typically made of metal or conductive materials and contact the scalp by means of adhesive bonding or clamping to record changes in electroencephalographic potential. Different numbers and layouts of EEG electrodes may be selected based on an experimental design and research requirements.

In other embodiments, in addition to the electroencephalography (EEG) acquisition electrode, other types of electrodes may also be used for brain signal acquisition, such as the electrocorticography (ECoG) acquisition electrode, the computed tomography (CT) electrode, and the deep brain stimulation (DBS) electrode. The ECoG electrode is an electrode that is directly implanted on the surface of the brain or placed under the dura mater. The ECoG can provide higher spatial resolution and signal quality, and can record more detailed neural activity. The CT electrode is an electrode used for positioning and navigation inside the brain tissue and are commonly used in the neurosurgery. The CT electrode can be accurately positioned using a computed tomography technology, allowing the brain signal acquisition in the specific areas. The DBS electrode is an electrode implanted deep within the brain and are used to treat the neurological disorders such as the Parkinson's disease and the depression. In addition to the stimulation function, the DBS electrode can also be used to record the brain signal to monitor the treatment efficacy.

In some embodiments, the electroencephalography EEG acquisition electrodes include the dry electrodes, which include electrode sheets and/or electrode needles. The electrode sheet is a flat metal sheet, typically made of a conductive material (such as silver/silver chloride). These electrode sheets can be placed on the surface of the scalp and in contact with the scalp to record the electroencephalographic activity. The electrode sheet can be adhered to the scalp and fixed using conductive glue or the electrode cap to ensure good contact. The electrode needle is a slender conductive needle, typically made of stainless steel or other conductive materials. The electrode needle can be directly inserted into the scalp to acquire the electroencephalography signal. When using the electrode needle, it is necessary to carefully follow hygiene and safety regulations and ensure that the electrode is placed and fixed correctly. The dry electrode has some advantages over other types of electrodes (such as the wet electrode), such as being easier to use, more comfortable, and not requiring additional conductive media (such as electrolytic gel).

In some embodiments, the electrodes disposed in at least one of the brain signal acquisition areas include the functional near-infrared spectroscopy FNIRS light source, the emitter, and the detector. The Functional Near-Infrared Spectroscopy (FNIRS) is a non-invasive neuroimaging technique used to measure the changes in the cerebral blood oxygen levels. The functional near-infrared spectroscopy (FNIRS) light source uses the near-infrared light source to emit the invisible light into the scalp tissue. The light source can use the infrared or the near-infrared light (with the wavelength ranging from 650 nanometers to 1000 nanometers), which can penetrate the skull and most soft tissues. The emitter is an assembly that directs the light from the light source to the measured area. The emitter may include one or more fiber optical bundles that direct the light emitted by the light source to the specific brain region. The detector is configured to measure the light reflected or transmitted back from the measured area and may be composed of the photosensitive detector (such as the photodiode) to measure the intensity of the reflected or transmitted light.

In some embodiments, the electrodes disposed in at least one of the brain signal acquisition areas include the anode electrode and the cathode electrode used for transcranial direct current stimulation (TDCS). The Transcranial Direct Current Stimulation (TDCS) is a technique that modulates the brain activity by delivering the weak direct current through the anode electrode and the cathode electrode placed on the scalp. In the TDCS, the anode electrode and the cathode electrode may be placed on the scalp to apply the current to the specific brain regions. The anode electrode, typically the positive electrode, enhances the current in the underlying brain region, thereby promoting the neuronal excitation. The cathode electrode, typically the negative electrode, inhibits the current in the underlying brain region, leading to the neuronal inhibition.

In some embodiments, as illustrated in FIG. 36, the wiring channel includes a horizontal wiring channel 81d and a vertical wiring channel 82d, which are configured to guide the signal transmission line connected to the brain signal mainboard card 61d to electrodes arranged at each brain signal acquisition area. Such a design allows the acquisition apparatus to comprehensively capture the brain signals from different brain regions, providing a more comprehensive and accurate brain signal input for subsequent data analysis and processing. Also, arrangement of the wiring channel also facilitates management and protection of the signal transmission line, enabling the entire apparatus to be more stable and reliable.

In some embodiments, the main body of the acquisition apparatus is constructed as a retractable structure or a dimension-adjustable structure to adapt to different head circumferences of the subjects. The wiring channel may be embedded in the main body.

In an exemplary embodiment of the present disclosure, the main body includes a second body 2d, a first body 1d, and a third body 3d. An acquisition area of the occipital lobe brain region is located at an inner side of the second body 2d, an acquisition area of the prefrontal lobe brain region and/or an acquisition area of the postfrontal lobe brain region are located at an inner side of the first body 1d, and an acquisition area of the parietal lobe brain region and/or an acquisition area of the temporal lobe brain region are located at an inner side of the third body 3d. In other embodiments, the acquisition area of the temporal lobe brain region may be located at two ends of the second body 2d, the first body 1d, and the third body 3d, corresponding to positions of two side brain regions of the human brain.

In some embodiments, as illustrated in FIG. 40, each body of the main body includes a third curved outer housing 51d, a third electrode group support 52d, and a third intermediate layer 53d. The third electrode group support 52d has a third electrode hole 521d for fixing the electrode. The third intermediate layer 53d is disposed between the third curved outer housing 51d and the third electrode group support 52d. The third electrode group support 52d is located at an inner layer of the third curved outer housing 51d. The third intermediate layer 53d (or a PCB plate) is elastic or is configured to mount wires that achieve electrical connection of the respective electrodes. Each electrode is mounted in the third electrode hole 521d of the third electrode group support 52d through an elastic member.

As illustrated in FIG. 36 to FIG. 40, the brain signal mainboard card 61d is disposed in the second body 2d, specifically at a junction of the second body 2d, the first body 1d, and the third body 3d. The brain signal mainboard card 61d and the occipital lobe brain signal acquisition area are located in the same second body 2d. Arrangement of the horizontal wiring channel 81d is relatively simple, which may utilize a cavity in the housing. Connection among the brain signal mainboard card 61d, the prefrontal lobe brain signal acquisition area, and the postfrontal lobe brain signal acquisition area is implemented by means of the horizontal wiring channel 81d passing through a connection structure between the second body 2d and the first body 1d. Connection between the brain signal mainboard card 61d and the parietal lobe brain signal acquisition area is implemented by means of the vertical wiring channel 82d passing through a connection structure between the second body 2d and the third body 3d. The horizontal direction and the vertical direction herein primarily refer to being located substantially in a same horizontal or vertical plane.

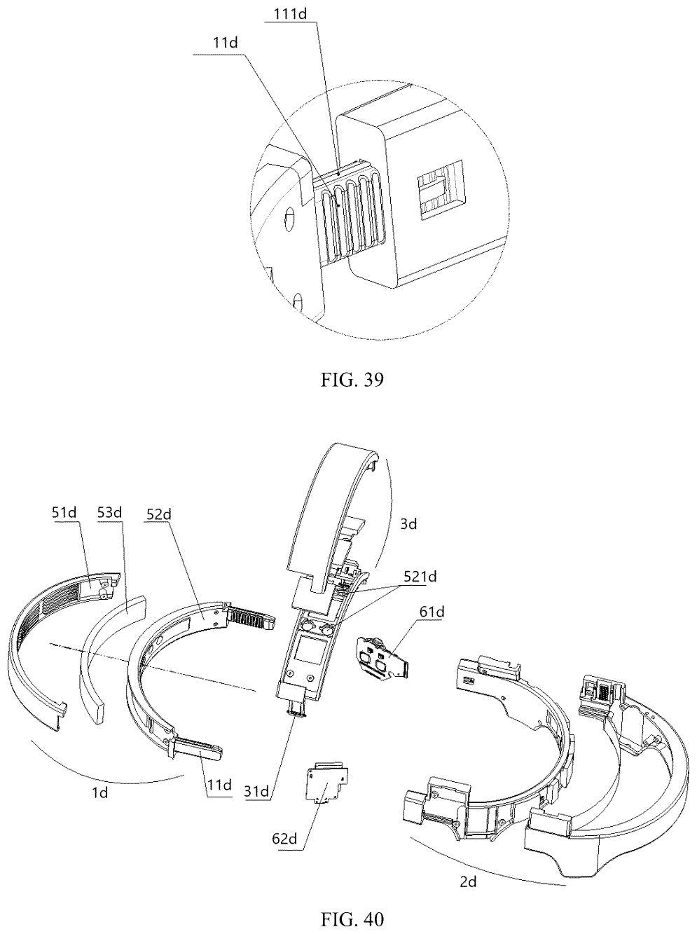

In the above embodiments, the second body 2d is fixedly arranged. As illustrated in FIG. 38, compared with the second body 2d, the first body 1d is retractably plugged laterally in front of the second body 2d. As illustrated in FIG. 39, the third body 3d is retractably plugged longitudinally above the second body. Part of the horizontal wiring channel 82d is formed in a plug-in structure of the first body 1d, and part of the vertical wiring channel 82d is formed in a plug-in structure of the third body 3d.

Such a design allows the acquisition apparatus to be adjusted based on the head circumference of the subject, improving the comfort and the adaptability. Also, embedding of the wiring channel inside the main body helps reduce clutter from external wires, protects the wires from being damaged, and also enhances overall aesthetics. This design provides greater flexibility and adaptability for the use of the acquisition apparatus, while also improving the comfort of the user and practicality.

In some embodiments, as illustrated in FIG. 36, FIG. 39, and FIG. 40, a plug-in structure between the horizontal wiring channel 81d and the first body 1d includes a first locking structure 11d. The first locking structure 11d has a through slot 111d, forming part of the horizontal wiring channel 81d. The first locking structure 11d may use an elastic locking tongue, which will not be repeated here. This design provides a mechanism for implementation of the horizontal wiring channel 81d through the first locking structure 11d and the through slot 111d. Arrangement of the slot 111d allows the wiring to pass through and be connected to a corresponding area smoothly. Also, the first locking structure 11d can effectively fix the first body 1d and the wiring channel, ensuring the head circumference to be adjustable.

In some embodiments, as illustrated in FIG. 36 and FIG. 40, the plug-in structure of the third body 3d includes a second locking structure 31d and a wire tube 32d that are separately arranged. The wire tube 32d is located at a side of the second locking structure 31d. The wire tube 32d has a hollow structure, forming part of the vertical wiring channel 82d. The second locking structure 31d may use an elastic locking tongue, which will not be repeated here. Such a design realizes arrangement of the vertical wiring channel 82d through the second locking structure 31d and the wire tube 32d. As a hollow structure, the wire tube 32d provides a channel for accommodating and guiding transmission of the line, while the second locking structure 31d can fix and adjust the third body 3d to ensure the head circumference to be adjustable.

In some embodiments, when the dry electrodes of the same form are adopted, the electrodes in the prefrontal lobe brain signal acquisition area, postfrontal lobe brain signal acquisition area, parietal lobe brain signal acquisition area, occipital lobe brain signal acquisition area, and temporal lobe brain signal acquisition area all adopt the third electrode sheet 41d or the third electrode needle 43d. In this way, it is indicated that regardless of which brain signal acquisition area the electrode group belongs to, the electrode forms are the same, which offers advantages in terms of uniform specifications, production convenience, and consistency control.

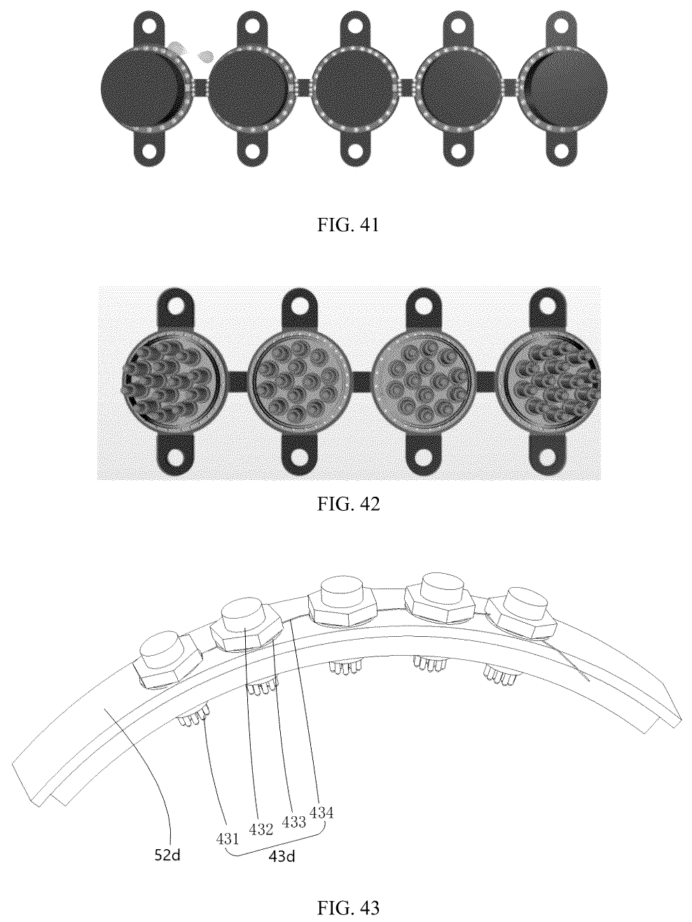

When the dry electrodes of different forms are adopted, at least part of the dry electrodes in the prefrontal lobe brain signal acquisition adopt the third electrode sheet 41d, and a shape of the electrode sheet is illustrated in FIG. 41. The dry electrodes in at least one of the postfrontal lobe brain signal acquisition area, the parietal lobe brain signal acquisition area, the occipital lobe brain signal acquisition area, and the temporal lobe brain signal acquisition area adopt the third electrode needle 43d, and a shape of the electrode needle is illustrated in FIG. 42. For example, areas with no hair or little hair, such as the prefrontal lobe brain region (or including the postfrontal lobe brain region), use the third electrode sheet 41d; while areas with dense hair, such as the parietal lobe brain region, the occipital lobe brain region, and temporal lobe brain region, use the third electrode needle 43d. Among the electrode groups at different locations, specific selection of the electrode form may vary, possibly due to differences in respective functional requirements or application scenarios.

In an embodiment of the present disclosure, the acquisition apparatus provides flexibility in the selection of dry electrode form to meet changing requirements for electrode form under different usage scenarios or needs, better serving the performance and applicability of the brain signal acquisition apparatus.

In another exemplary embodiment of the present disclosure, the third electrode needle 43d may be a third electrode needle 43d without an elastic structure. In another exemplary embodiment of the present disclosure, the third electrode needle 43d may be mounted at the brain signal acquisition area of the main body through the elastic member. In another exemplary embodiment of the present disclosure, the third electrode needle 43d may have an elastic structure. The third electrode needle 43d includes a needle barrel, a needle tip with a rebound stroke, and an elastic member disposed inside the needle barrel for rebounding the needle tip.

In some embodiments, when the dry electrode is disposed in an integrated structure inside the housing of the brain signal acquisition apparatus, the dry electrode needs to be properly fixed. Regarding methods for fixing the dry electrode, a variety of different fixing methods are determined mainly based on different electrode forms.

The active electrode and the passive electrode are two types of electrodes commonly used in the field of biosignal acquisition. A main difference between the active electrode and the passive electrode is whether an amplifier or a gain device is provided. The active electrode contains a built-in amplifier or the gain device, which can amplify the signal when coming into contact with a biosignal. This type of electrode is usually used to amplify and acquire weak biosignals, such as the electroencephalography (EEG) or electromyography (EMG). The active electrode can effectively reduce the interference of the cables or transmission media on the signal, and can provide a higher signal-to-noise ratio. The passive electrode does not have the built-in amplifier or the gain device, and only serves as a carrier for transmitting the biosignals. Therefore, signals acquired by the passive electrode are relatively small and need to be amplified in subsequent signal processing. This type of electrode is usually used for general biosignal acquisition, such as electrocardiogram (ECG) or biopotential measurement. The passive electrode is usually simpler, more cost-effective, and easier to maintain and manage.

For example, when the dry electrode is fixed to the brain signal acquisition area of the main body: in a case where the dry electrode includes the third electrode sheet 41d of the passive electrode, the third electrode sheet 41d is fixedly soldered to a PCB board, and then the PCB board is fixed inside the main body through a connection member; in a case where the dry electrode includes the third electrode needle 43d of the passive electrode, the third electrode needle 43d of the passive electrode is fixedly soldered to the PCB board, and then the PCB board is fixed inside the main body through the connection member. In another exemplary embodiment of the present disclosure, as illustrated in FIG. 43, a threaded post 432 is disposed at a tail end of the electrode needle of the passive electrode. The threaded post 432 is inserted into the third electrode hole 521d of the third electrode group support 52d in the brain signal acquisition area, and a washer 433 with a signal transmission line 434 is fixed to the threaded post 432 using a nut 433 inside the main body.

In a case where the dry electrode includes the third electrode sheet 41d of the active electrode or the third electrode needle 43d of the active electrode, the third electrode sheet 41d of the active electrode or the third electrode needle 43d of the active electrode is fixedly soldered to a side of the PCB board. A processing circuit is placed on the other side of the PCB board, and then the PCB board is fixed inside the main body through the connection member.

What is claimed is:

1. An electroencephalography dry electrode, comprising a substrate and a plating layer, wherein:
the substrate comprises a base and a plurality of columnar members;
an end of each of the plurality of columnar members is fixed to the base;
at least a surface of each of the plurality of columnar members is provided with the plating layer; and
the plating layer on the surface of each of the plurality of columnar members is electrically connected to the base.

2. The electroencephalography dry electrode according to claim 1, wherein each of the plurality of columnar members comprises a first member, a second member, and a first elastic member, wherein:
the first member is sleeved at the second member;
the second member is configured to move relative to the first member in an axial direction;
the first elastic member is disposed in the first member;
two ends of the first elastic member are respectively connected to the first member and the second member; and
the first elastic member is configured to apply a restoring force to the second member to move outward from the first member.

3. The electroencephalography dry electrode according to claim 2, wherein each of the plurality of columnar members comprises a copper member.

4. The electroencephalography dry electrode according to claim 3, wherein the second member has a recessed cavity, wherein:
the recessed cavity has a first opening facing towards the first elastic member;
the first elastic member passes through the first opening with one end of the first elastic member located in the recessed cavity; and
the second member is formed by stamping and stretching.

5. The electroencephalography dry electrode according to claim 4, wherein:
a limit portion is disposed at a circumferential edge of the first opening, and has a dimension greater than an outer diameter of the second member in a direction perpendicular to an axis direction; and
the first member comprises a tube body, wherein:
both ends of the tube body have an opening;
an inner diameter of an edge of the opening of the tube body adjacent to the second member is smaller than the dimension of the limit portion in the direction perpendicular to the axis direction;
the limit portion is disposed in the tube body; and
the tube body is integrally formed by stamping, stretching and blanking.

6. The electroencephalography dry electrode according to claim 1, wherein:
a surface of the base is provided with the plating layer;
the plating layer on the surface of the base is electrically connected to the plating layer on the surface of the columnar member; and
the base is integrally injection-molded with the plurality of columnar members.

7. The electroencephalography dry electrode according to claim 6, further comprising a conductive sheet, wherein:
the base has a connection hole configured to be connected to a connection member, to fix the conductive sheet to the substrate; and
the conductive sheet is electrically conductive with the plating layer on the surface of the base.

8. The electroencephalography dry electrode according to claim 6, further comprising a second elastic member and a mounting member, wherein:
the second elastic member is connected to a side of the base away from the columnar member;
the second elastic member is extensible and retractable in an axial direction of the columnar member; and
the second elastic member is connected between the base and the mounting member.

9. The electroencephalography dry electrode according to claim 8, further comprising a guide plate, wherein:
the guide plate is disposed around an outer circumference of the second elastic member; and
at least one of the base and the mounting member is connected to the guide plate via a guide structure, the guide structure comprising a guide groove and a guide portion movably inserted into the guide groove in the axial direction of the columnar member.

10. An electroencephalography measurement apparatus comprising an electroencephalography dry electrode, the electroencephalography dry electrode comprising a substrate and a plating layer, wherein:
the substrate comprises a base and a plurality of columnar members;
an end of each of the plurality of columnar members is fixed to the base;
at least a surface of each of the plurality of columnar members is provided with the plating layer; and
the plating layer on the surface of each of the plurality of columnar members is electrically connected to the base.

11. The electroencephalography measurement apparatus according to claim 10, further comprising a wearable assembly and a pushing assembly, wherein:
the wearable assembly is adapted to be worn on a head of a user, and has a mounting cavity where the pushing assembly is located;
the electroencephalography dry electrode is movably connected to the wearable assembly; and
the pushing assembly is configured to push the electroencephalography dry electrode to move towards the head, when a contact impedance between the electroencephalography dry electrode and the head is greater than or equal to a first impedance threshold.

12. The electroencephalography measurement apparatus according to claim 11, wherein the pushing assembly is configured to push, at a reference pushing intensity, the electroencephalography dry electrode to move towards the head when the contact impedance between the electroencephalography dry electrode and the head is greater than or equal to the first impedance threshold, wherein:
the reference pushing intensity is determined based on the contact impedance and a correspondence between an impedance and an intensity; and
the correspondence records a plurality of impedances, with any two of the plurality of impedances being different.

13. The electroencephalography measurement apparatus according to claim 10, further comprising a main body, and a frontal lobe electrode group and a non-frontal lobe electrode group that are disposed at the main body, wherein:
the frontal lobe electrode group comprises a plurality of sheet-shaped dry electrodes mounted at a region of the main body corresponding to a frontal lobe brain region of a user; and
the non-frontal lobe electrode group comprises a plurality of needle-shaped dry electrodes mounted at a region of the main body corresponding to a non-frontal lobe brain region of the user, wherein the frontal lobe brain region comprises at least one of a prefrontal lobe brain region and a postfrontal lobe brain region, and the non-frontal lobe brain region comprises at least one of a parietal lobe brain region, an occipital lobe brain region, and a temporal lobe brain region.

14. The electroencephalography measurement apparatus according to claim 10, further comprising at least two electrode group modules corresponding to different brain regions, wherein:
each of the at least two electrode group modules has a rigid semi-ring structure, the at least two electrode group modules being connected with each other to form an integrated structure;
at least one of the at least two electrode group modules has a retractable structure, enabling the module to move in a direction towards or away from a center of the module to adjust a dimension of the module; and
the brain regions comprise a prefrontal lobe brain region, a postfrontal lobe brain region, a parietal lobe brain region, an occipital lobe brain region and/or a temporal lobe brain region.

15. The electroencephalography measurement apparatus according to claim 14, wherein the at least two electrode group modules comprise at least two of a first electrode group module corresponding to the prefrontal lobe brain region and/or the postfrontal lobe brain region, a second electrode group module corresponding to the occipital lobe brain region, and a third electrode group module corresponding to the parietal lobe brain region and/or the temporal lobe brain region.

16. The electroencephalography measurement apparatus according to claim 15, wherein a first plug-in structure is formed at a connection part of the first electrode group module and the second electrode group module, wherein at least one end of one module of the first electrode group module and the second electrode group module is formed with a protruding first plug-in block, and a corresponding end of the other module of the first electrode group module and the second electrode group module has a recessed first plug-in groove, the first plug-in block being configured to move in the first plug-in groove to achieve extension and retraction.

17. The electroencephalography measurement apparatus according to claim 10, further comprising a main body, and a brain signal mainboard card, a plurality of electrodes, and a signal transmission line that are integrally disposed at the main body, wherein:
the main body has an integrated rigid structure having a wiring channel formed inside or at a surface of the main body;
the plurality of electrodes are grouped and arranged in at least one brain signal acquisition area of the main body;
the wiring channel of the main body extends from the brain signal acquisition area to an area of the main body for mounting the brain signal mainboard card, and is configured to embed the signal transmission line; and
the signal transmission line is configured to connect the plurality of electrodes and the brain signal mainboard card, to transmit a brain signal acquired by the plurality of electrodes.

18. The electroencephalography measurement apparatus according to claim 17, wherein:
the brain signal acquisition area of the main body comprises one or more of a prefrontal lobe brain signal acquisition area, a postfrontal lobe brain signal acquisition area, a parietal lobe brain signal acquisition area, an occipital lobe brain signal acquisition area, and a temporal lobe brain signal acquisition area; and
the wiring channel comprises a horizontal wiring channel and a vertical wiring channel, which are configured to guide the signal transmission line connected to the brain signal mainboard card to electrodes arranged at each brain signal acquisition area.

19. The electroencephalography measurement apparatus according to claim 18, wherein the electrodes arranged in at least one of the brain signal acquisition areas comprise an anode electrode and a cathode electrode used for transcranial direct current stimulation (TDCS).

20. The electroencephalography measurement apparatus according to claim 19, wherein:
the wiring channel is embedded in the main body; and
the main body comprises a first body, a second body, and a third body, wherein:
an acquisition area of an occipital lobe brain region is located at an inner side of the second body, an acquisition area of a prefrontal lobe brain region and/or an acquisition area of a postfrontal lobe brain region are located at an inner side of the first body, and an acquisition area of a parietal lobe brain region and/or an acquisition area of a temporal lobe brain region are located at an inner side of the third body;
the second body is fixedly arranged, with the first body being retractably plugged laterally in front of the second body, and the third body being retractably plugged longitudinally above the second body; and
the brain signal mainboard card is disposed in the second body, part of the horizontal wiring channel is formed in a plug-in structure of the first body, and part of the vertical wiring channel is formed in a plug-in structure of the third body.
